(19) United States
(12) Patent Application Publication
(10) Pub. No.: US 2024/0347179 A1
(43) Pub. Date: Oct. 17, 2024

(54) Systems and Methods for Medical Instrument Management

(71) Applicant: Innovative Perioperative Technologies, LLC, Portsmouth, NH (US)

(72) Inventors: Christine Wall, Lynbrook, NY (US); David Nichols, Dover, NH (US); Kevin Fedigan, New York, NY (US); Walter J. Oko, Woodbridge, CT (US)

(73) Assignee: Innovative Perioperative Technologies, LLC, Portsmouth, NH (US)

(21) Appl. No.: 18/134,907

(22) Filed: Apr. 14, 2023

(51) Int. Cl. G16H 40/20; G06F 3/0484

(52) U.S. Cl. CPC G16H 40/20; G06F 3/0484

(57) ABSTRACT

A system for medical instrument management is provided that includes one or more graphical user interfaces displaying a number of trays needed to be sourced for a surgery, a number of trays at the facility, sourced for a surgery, and needed to be sent through a sterilizer, and a number of trays at the facility, sourced for a surgery, and sent through the sterilizer. The system further includes one or more tags configured to transmit location information and one or more processors configured to receive surgery information for a surgery at the facility entered into the one or more graphical user interfaces, where the surgery information includes a number of trays that need to be sourced to the facility for the surgery. The one or more processors are configured to update graphical user interface(s) associated with the system to display a number of the one or more trays that have been sourced for the surgery and need to be sent through the sterilizer. A method for medical instrument management is also provided.

### BACKGROUND

Providing high quality and patient-centered health care is becoming increasingly dependent on the efficiency of medical delivery supply chains and operations, as well as effective and timely management of medical instruments and information. Before a medical device has any contact with a patient, it must traverse an exceedingly complex and opaque supply chain. In addition, limited efforts to capture and/or access comprehensive medical device information at the point-of-care (POC) and across organizations have also been a barrier to tracking medical device utilization patterns.

### SUMMARY

A system for medical instrument management is provided. The system includes one or more graphical user interfaces displaying a number of trays needed to be sourced for a surgery, a number of trays at the facility, sourced for a surgery, and needed to be sent through a sterilizer, and a number of trays at the facility, sourced for a surgery, and sent through the sterilizer. The system further includes one or more tags configured to transmit location information. The system also includes one or more processors configured to receive surgery information for a surgery at the facility entered into the one or more graphical user interfaces, where the surgery information includes a number of trays that need to be sourced to the facility for the surgery. The one or more processors are configured to update the one or more graphical user interfaces to display the number of trays that need to be sourced to the facility for the surgery. The one or more processors are configured to receive a notification that one or more trays have been sourced for the surgery. The one or more processors are configured to receive one or more tag identifiers associated with the one or more tags assigned to the one or more trays. The one or more processors are configured to update the graphical user interface to display a number of the one or more trays that have been sourced for the surgery and need to be sent through the sterilizer. The one or more processors are configured to receive temperature information from the one or more tags assigned to the one or more trays indicating that the one or more trays have been sent through the sterilizer. The one or more processors are configured to update the graphical user interface to display a number of the one or more trays that have been sourced for the surgery and sent through the sterilizer.

A method for medical instrument management is provided. The method includes receiving surgery information for a surgery at the facility entered into one or more graphical user interfaces, wherein the surgery information includes a number of trays that need to be sourced to the facility for the surgery. The method includes updating the one or more graphical user interfaces to display the number of trays that need to be sourced to the facility for the surgery. The method includes receiving a notification that one or more trays have been sourced for the surgery. The method includes receiving one or more tag identifiers associated with one or more tags assigned to the one or more trays. The method includes updating the graphical user interface to display a number of the one or more trays that have been sourced for the surgery and need to be sent through a sterilizer. The method includes receiving temperature information from the one or more tags assigned to the one or more trays indicating that the one or more trays have been sent through the sterilizer. The method includes updating the graphical user interface to display a number of the one or more trays that have been sourced for the surgery and sent through the sterilizer.

### BRIEF DESCRIPTION OF THE DRAWINGS

To assist those of skill in the art in making and using the described system and associated methods, reference is made to the accompanying figures. The accompanying figures, which are incorporated in and constitute a part of this specification, illustrate one or more embodiments of the invention and, together with the description, help to explain the invention. Illustrative embodiments are shown by way of example in the accompanying drawings and should not be considered as limiting. In the figures:

FIG. 1 is a block diagram illustrating a system for medical instrument and information management, according to an exemplary embodiment.

FIG. 2 is a block diagram illustrating a system for medical instrument and information management, according to an exemplary embodiment.

FIG. 3 illustrates an exemplary pre-operative dashboard user interface displaying a list of surgeries that require one or more surgical trays according to an exemplary embodiment.

FIG. 4 illustrates an exemplary dashboard user interface for generating and/or scheduling a new surgery.

FIG. 5 illustrates an exemplary dashboard user interface for adding trays to the new surgery identified in FIG. 4, according to an exemplary embodiment.

FIG. 6 illustrates an exemplary dashboard user interface for sourcing and/or checking in trays for a surgery, according to an exemplary embodiment.

FIG. 7 illustrates an exemplary dashboard user interface with a pop-up window for entering a reason for a late delivery of trays, according to an exemplary embodiment.

FIG. 8 illustrates an exemplary dashboard user interface displaying trays sourced for a surgery, according to an exemplary embodiment.

FIG. 9 illustrates an exemplary dashboard user interface displaying a summary of the trays displayed in FIG. 8, according to an exemplary embodiment.

FIG. 10 illustrates an exemplary dashboard user interface displaying a window displaying information associated with a tray selected in FIG. 9, according to an exemplary embodiment.

FIG. 11 illustrates an exemplary dashboard user interface displaying a progress window associated with the tray selected in FIG. 9, according to an exemplary embodiment.

FIG. 12 illustrates an exemplary dashboard user interface displaying a history window associated with the tray selected in FIG. 9, according to an exemplary embodiment.

FIG. 13 illustrates an exemplary dashboard user interface displaying a mapping window associated with the tray selected in FIG. 9, according to an exemplary embodiment.

FIG. 14 illustrates an exemplary pre-operative dashboard user interface displaying a list of surgeries that required one or more surgical tray, according to an exemplary embodiment.

FIG. 15 illustrates an exemplary pre-operative dashboard user interface illustrating notifications transmitted to one or more parties based on actions performed by activities triggered by actions through the dashboards, according to an exemplary embodiment.

FIG. 16 illustrates an exemplary pre-operative dashboard user interface in which an administer for the facility can acknowledge or deny accepting trays for a surgery, according to an exemplary embodiment.

FIG. 17 illustrates an exemplary pre-operative dashboard user interface in which an administer for the facility has denied accepting a tray for a surgery using the dashboard user interface shown in FIG. 16, according to an exemplary embodiment.

FIG. 18 illustrates an exemplary pre-operative dashboard user interface displaying a window in which the administer for the facility can enter a reason for denying the tray for the surgery as shown in FIG. 17, according to an exemplary embodiment.

FIG. 19 illustrates an exemplary pre-operative dashboard user interface in which an administer for the facility has acknowledged receiving trays for a surgery using the dashboard user interface shown in FIG. 16, according to an exemplary embodiment.

FIG. 20 illustrates an exemplary pre-operative dashboard user interface corresponding to the pre-operative dashboard user interface of FIG. 14 updated to reflect that one tray for the surgery has been denied, as shown in FIG. 17, according to an exemplary embodiment.

FIG. 21 illustrates an exemplary dashboard user interface displaying a list of tags, according to an exemplary embodiment.

FIG. 22 illustrates an exemplary dashboard user interface displaying locations of assigned tags based on a search tray name (“Flex Express”), according to an exemplary embodiment.

FIG. 23 illustrates an exemplary window displaying details associated with a tag shown in FIG. 22, according to an exemplary embodiment.

FIG. 24 illustrates an exemplary window displaying a progress bar for a tag shown in FIG. 22, according to an exemplary embodiment.

FIG. 25 illustrates an exemplary post-operative dashboard user interface illustrating post-surgery trays, according to an exemplary embodiment.

FIG. 26 illustrates an exemplary post-operative dashboard user interface displaying post-operative trays associated with a surgery performed a physician, according to an exemplary embodiment.

FIG. 27 illustrates an exemplary post-operative dashboard user interface displaying a tray that has been checked out using the dashboard user interface shown in FIG. 26, according to an exemplary embodiment.

FIG. 28 illustrates an exemplary mobile application user interface displaying a dashboard user interface displaying selectable graphics associated with pre-operative actions, post-operative actions, completed, and cancelled, according to an exemplary embodiment.

FIG. 29 illustrates an exemplary mobile application user interface displaying a dashboard user interface displaying trays to be sourced based on facilities, according to an exemplary embodiment.

FIG. 30 illustrates an exemplary mobile application user interface displaying a dashboard user interface displaying trays to be sourced, filtered based on the trays, the invocation of a mobile phone scanning software invoked by the camera icon as shown in FIG. 28, according to an exemplary embodiment.

FIG. 31 illustrates an exemplary mobile application user interface displaying a dashboard user interface displaying trays to be sourced filtered based on the surgery represented in FIG. 30 with the option to remove a selected tray, according to an exemplary embodiment.

FIG. 32 illustrates an exemplary mobile application user interface for scanning a visual identifier to check in one or more trays and/or checkout one or more trays, according to an exemplary embodiment.

FIG. 33 illustrates an exemplary mobile application user interface displaying a list of scanned trays at a location, with options for different user action, according to an exemplary embodiment.

FIG. 34 illustrates an exemplary mobile application user interface generated after FIG. 33, designed to prompt the user to take action for trays ready for pick up, according to an exemplary embodiment.

FIG. 35 illustrates an exemplary mobile application user interface displaying a map of locations of facilities in which a representative has trays checked into, according to an exemplary embodiment.

FIG. 36 illustrates an exemplary dashboard user interface for viewing consigned trays, according to an exemplary embodiment.

FIG. 37 illustrates an exemplary user interface for adding a consigned tray, according to an exemplary embodiment.

FIG. 38 illustrates an exemplary user interface for checking in a consigned tray, according to an exemplary embodiment.

FIG. 39 illustrates an exemplary user interface for checking out a consigned tray, according to an exemplary embodiment.

FIG. 40 illustrates an exemplary user interface for reviewing and printing a label after checking in a consigned tray, according to an exemplary embodiment.

FIG. 41 illustrates an exemplary pre-operative dashboard user interface (similar to FIG. 3) displaying a list of surgeries that require one or more surgical trays, according to an exemplary embodiment.

FIG. 42 illustrates an exemplary pre-operative dashboard user interface to add a new surgery, according to an exemplary embodiment.

FIG. 43 illustrates an exemplary user interface to add a tray to a surgery by scanning a visual identifier associated with the tray, according to an exemplary embodiment.

FIG. 44 illustrates an exemplary dashboard user interface after a tray is added to a surgery, according to an exemplary embodiment.

FIG. 45 illustrates an exemplary dashboard user interface illustrating an option 4502 to turn over a tray, according to an exemplary embodiment.

FIG. 46 illustrates an exemplary user interface for turning over a tray, according to an exemplary embodiment.

FIG. 47 illustrates an exemplary user interface for reviewing and printing a label after turning over a tray, according to an exemplary embodiment.

FIG. 48 illustrates an exemplary user interface displaying a list of unassigned trays for completed surgeries

FIG. 49 schematically depicts an example computer system that can be used to execute some embodiments of the present disclosure.

FIG. 50 is a photograph of a tray with a tag located within the tray, in accordance with an exemplary embodiment.

FIG. 51 is a flow chart of an example process that can be executed in accordance with some embodiments of the present disclosure.

### DETAILED DESCRIPTION OF EMBODIMENTS

An exemplary embodiment provides advantageous systems and methods that enable improved instrument and information management that promotes efficient medical delivery supply chains and operations. The described systems and methods facilitate tracking of medical instruments and other medical supplies (e.g., pharmaceuticals, gowns, bandages, etc.) associated with a medical facility.

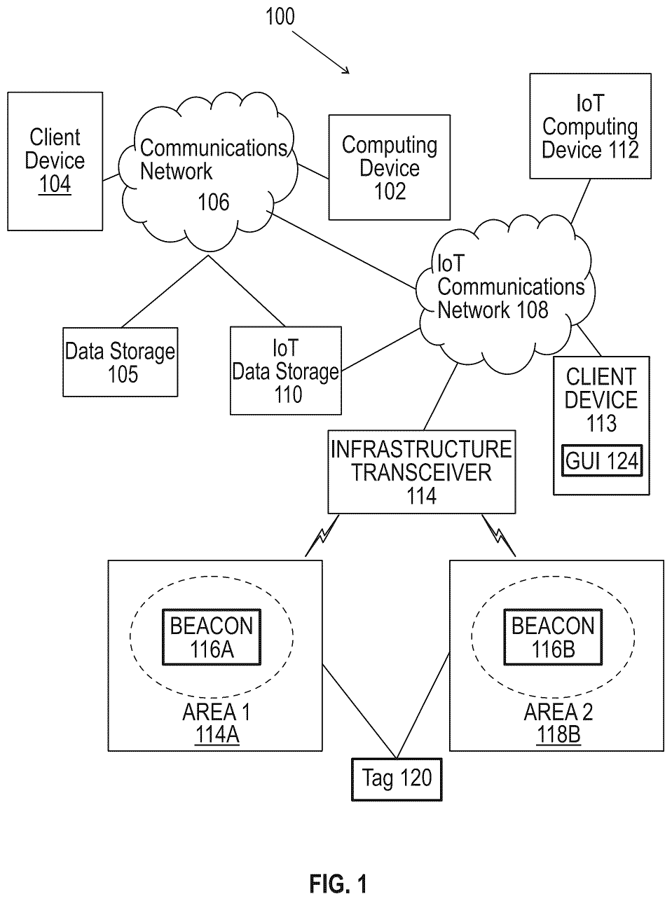

FIG. 1 is a block diagram illustrating a system 100 for medical instrument and information management, according to an exemplary embodiment. The system 100 includes one or more internet of things (IoT) communication networks 108, one or more IoT storage devices 110, one or more IoT computing devices 112, one or more client devices 113, one or more infrastructure transceivers 114, one or more beacons 116A, 116B corresponding to particular areas (beacons 116A, 116B corresponding to areas 118A, 118B, respectively, are shown in the figure for illustrative purposes), and one or more tags (tag 120 is shown in the figure for illustrative purposes). In further embodiments, there may be additional beacons, areas, and tags.

In some embodiments, system 100 further includes one or more computing devices 102, one or more client devices 104, one or more storage devices 105, and one or more communication networks 106. These components are described in U.S. Non-Provisional patent application Ser. No. 17/146,251, the entire content of which is incorporated herein by reference. In some embodiments, IoT computing device 112 can access data from storage devices 105.

In some embodiments, IoT computing devices 112 can be a physical computing device, a virtual computing device, or multiple physical and virtual computing devices functioning as one computing device. In some embodiments, the IoT computing devices 112 can be a cloud-based system providing computational functionality remote from the facility where the system is deployed. In some embodiments, the IoT computing devices 112 can operate on a software stack including an operating system, including but not limited to Microsoft Windows-based distributions and *nix-based distributions.

Some embodiments include additionally software support for the operation of the IoT computing devices 112. Software support can include library dependencies including network stacks implementing portions or all of the Open Systems Interconnection (OSI) network model. Additionally, software support can include libraries supporting the creation and reception of Internet of Things (IoT) data including but not limited to IoT platforms such as IoTivity and Zephyr. The IoT computing devices 112 may include modules including an application or a suite of applications coded to operate in the environment provided by the IoT computing devices 112. These modules can be implemented but not limited to C++, JAVA, and C#.

The network 108 interface can be utilized in embodiments of the system. In some embodiments, the network 108 can be a wide area network (WAN) or the Internet. The network 108 can be operable to transport data packets compatible with the infrastructure transceivers 109. In some embodiments, compatible data packets can include data packets with transmission control protocol (TCP) or user datagram protocol (UDP) routing information, as well as an accessible application layer. The network 108 can interface with the computing device 102, the client device 104, IoT computing device 112, client device 113, storage device 105, IoT storage device 110, infrastructure transceiver 114, and other networks. The network 106 and/or network 108 can be a combination of wired and wireless connection inclusively.

A client device 113 can be attached to the network 108. The client device 113 can include a graphical user interface (GUI) 124 executing on the client device 113. In some embodiments, the client device 113 can be implemented at a personal computing device, such as a smart phone, tablet, laptop, or desktop computer. The client device 113 can have network interface functionality so that it can interface with the network 108. The client device 113 can operate on a software stack including an operating system including but not limited to Microsoft Windows-based distributions, Apple iOS distributions and *nix-based distributions, including the Android operating system. The client device 113 can include support software stacks to implement graphical elements of the GUI 124. The GUI 124 can be implemented in common programming languages, including but not limited to JAVA, Angular.js, and Objective-C, and APIs for interacting with client device 113 subsystems.

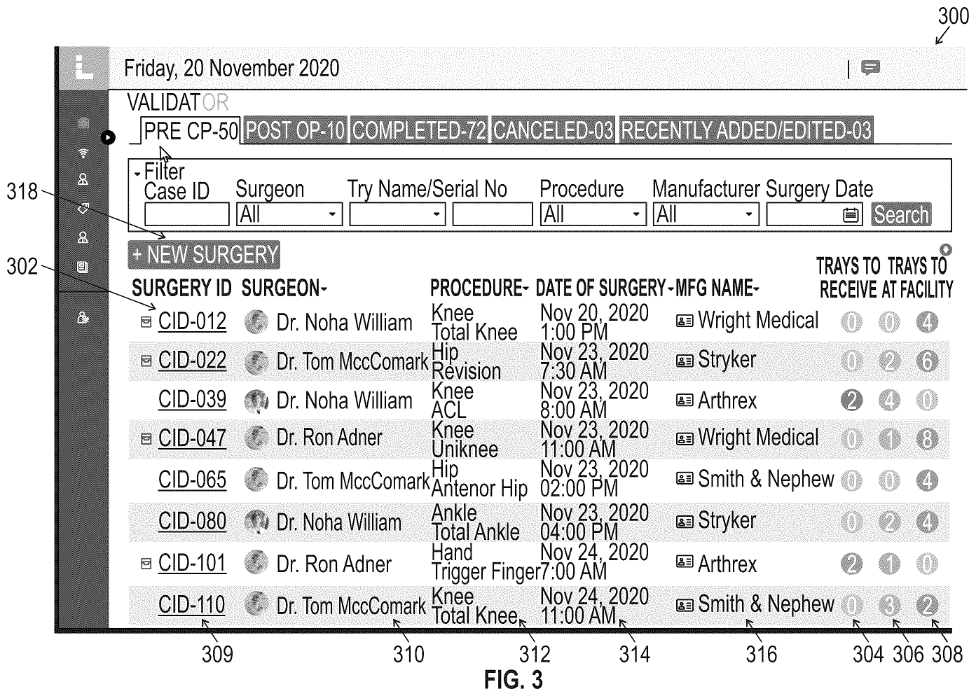

The GUI 124 can be configured to display the graphical user interface dashboards as shown in FIGS. 3-48. For example, the dashboards as shown in FIGS. 3-48 may be displayed within an downloadable application, a website, and/or a smartphone. Additionally, the GUI 124 can be configured to display an indication of parametric sterilization release and/or the location of a hardened tag affixed to a medical supply (e.g., a medical tray) within a facility.

One or more infrastructure transceiver 114 can be connected to the network 108. The infrastructure transceiver 114 can receive signals from beacons 116A, 116B throughout a facility. The infrastructure transceiver 114 can connect to beacons 116A, 116B utilizing a wireless connection as illustrated here, however wired connections are inclusive to some embodiments. The infrastructure transceiver 114 provides an interface for the beacons 116A, 116B to communication through the network 108 to the IoT computing device 112. The infrastructure transceiver 114 can utilize wireless protocols including but not limited to Bluetooth, Zigbee, WiFi, and Wideband cellular connections for IoT devices (e.g. NB-Iot) to communicate with the beacons 116A, 116B.

While the described systems and methods herein recite associating a tag 120 with a medical tray, in other embodiments the tag 120 may be used with other medical instruments and/or medical supplies, such as pharmaceuticals, gowns, bandages, etc.

An exemplary embodiment also describes the use of tags used to monitor/track instruments/medical equipment within a facility. The disclosed tags may include functionalities that permit and/or support location-based validation of autoclave parametric sterilization release (PSR) information/metrics, and/or communication of such PSR information/metrics to communication networks for processing and/or data access.

One or more beacons 116A, 116B can correspond to one or more areas 118A, 118B within a facility. The beacons 116A, 116B can be implemented utilizing a near field communication technology including but not limited to Bluetooth LE. The beacons 116A, 116B can be connected to the infrastructure transceiver 114 utilizing the same communication technology for tag detection or a different communication technology. For example, the beacons 116A, 116B can interface with the tag 120 utilizing Bluetooth LE and additionally connect with the infrastructure transceiver 114 utilizing a WiFi connection. The beacons 116A, 116B can be implemented upon many different platforms including but not limited to iBeacon and Eddystone.

In some embodiments, the tag 120 can be a near field communication device that operates in a low power envelope. In an exemplary embodiment, the tag 120 is a Bluetooth® tag used in Real-Time Location Systems (RTLS). As the tag 120 comes within the broadcast range of the beacon 116A, the tag 120 and the beacon 116A present a handshake. The beacon 116A identifies the tag and maintains a connection with the tag 120 utilizing periodic polling. The beacon 116A communicates with the infrastructure transceiver 114 notifying the IoT computing device 112 through the network as to which beacon 116A the tag 120 is currently associated. The IoT computing device 112 correlates the location of the beacon 116A with an area 116A within a facility and determines that the tag 120 is located in area 1. In some embodiments, the area may be a room or a department. In some embodiments, each room or department may include a beacon or multiple beacons to track the tag 120.

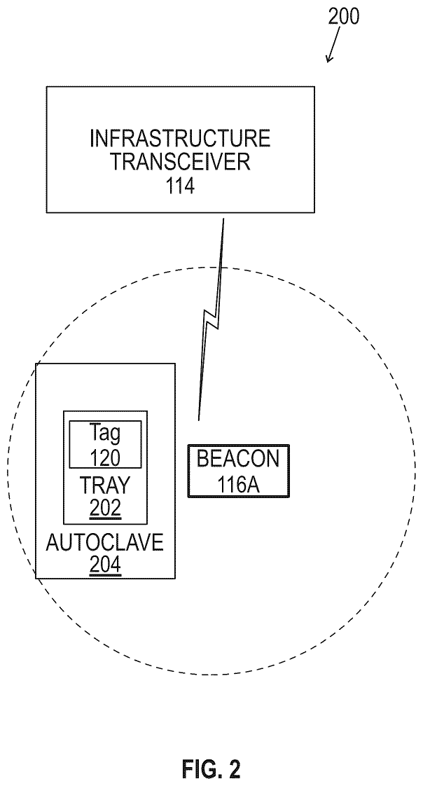

FIG. 2 is a block diagram illustrating a system 200 for medical instrument and information management, according to an exemplary embodiment. In some embodiments, the system 200 is the same as described in FIG. 1.

As illustrated in the system 200, tag 120 can be integrated or affixed to a medical instrument tray 202 and placed in an sterilizer 204, such as an autoclave 204 or a washer 204. The autoclave 204 is a machine used to sterilize equipment using elevated temperature and/or pressure. The tag 120 includes a hardened shell to survive repeated autoclave 204 cycles while not deteriorating in longevity or performance as a result of the rigors of the autoclave cycles. Internal to the shell can be a microcontroller based on IoT designs. The microcontroller hosts firmware and software for the operation of the near field communication transceiver as well as an array of sensors. The sensors can include thermometers and pressure sensors. The sensors can physically be integrated into the hardened shell, or internal to the shell. A battery can be included within the hardened shell to power the microprocessor and sensors. As such, the battery can be designed to meet the rigors of the autoclave 204 process as well. To conserve power, the microcontroller can deactivate the sensors when the tag 120 has not received a signal from a designated beacon 116A.

In some embodiments, when the tag 120 receives a near field communication from a designated beacon 116A, the microcontroller can take interval sampling data from the sensors. As long as the tag 120 detects the designated beacon 116A, the interval sampling data will be accumulated. Upon a timer threshold expiration without a handshake with the designated beacon 116A, the tag 120 can stop the interval sampling data of the sensors.

In some embodiments, tag 120 can report adequate or inadequate sterilization to the designated beacon 116A. In some embodiments, the IoT computing device 112 analyzes the above described information and provides a notification alert and/or other signaling communication to the client device 113 if the sterilization process failed to satisfy required standards/thresholds (e.g., to prevent the product from being transported to the operating room or other patient-related setting). In some embodiments, the IoT computing device 112 analyzes the above described information and provides a notification alert and/or other signaling communication to the client device 113 if the sterilization process satisfies required standards/thresholds.

Alternatively, the tag 120 can stop the interval sampling data of the sensors when the tag 120 handshakes with a non-designated beacon.

In another embodiment, the tag 120 collects interval sampling data continuously. The interval sampling data can be transmitted from the tag 120 to the beacon 116A and then to the infrastructure transceiver 114. Once the interval sampling data is received by the infrastructure transceiver 114, the sampling data is packetized and propagated through the network 108 to the IoT computing device 112. In some embodiments, the sampling data is propagated from the IoT computing device 112 through the network 108 to the client device 113 for display via GUI 124.

The IoT computing device 112 receives interval sampling data through interfaces provided by the IoT computing device 112. The interfaces can be defined within the operating system and additional software stack executing on the IoT computing device 112. The IoT computing device 112 can receive a dataset of interval sampling data and process it accordingly. The interval sampling data can include but not be limited to temperature data, pressure data, time data, and location data. The interval sampling data can be correlated to a time-based recording ledger, where for every time entry in the dataset, there is a corresponding temperature, pressure, and location entry. Alternatively, for efficiency and to reduce data duplication, the time-based recording ledger can include entries based on time with corresponding temperature and pressure entries, but only include the location entry when the combination reporting from a beacon 116A, 110B, and a tag 120 indicate a change in location.

FIG. 3 illustrates an exemplary pre-operative dashboard user interface 300 displaying a list of surgeries requiring one or more surgical or medical device trays, according to an exemplary embodiment. In some embodiments, the interface 300 enables medical/surgical representatives as well as facility employees to manage trays. The interface 300 includes a list of entries 302, including relevant information, such as, but not limited to, a surgery identifier 309, a name of a surgeon performing the surgery 310, a name of a procedure 312, a name of a sub-procedure, a date and time of the surgery 314, and a name of a manufacturer associated with the one or more trays 316. In some embodiments, the interface 300 includes additional information, such as a name of a facility where the surgery is being performed or other notes input during check in.

Each entry 302 is further associated with a number 304 of trays required for the surgery but have not been received by the facility. Each entry 302 further includes a number 306 of trays required for a surgery and received by the facility but not yet sent through the sterilizer and ready for surgery. Each entry 302 further includes a number of 308 of trays required for a surgery and that have been through the sterilizer and are ready for surgery. The numbers 304, 306, 308 are updated as trays are received by the facility and sent through the sterilizer for surgery. For example, as a tray required for a surgery is received by a facility, the number 304 of trays required for the surgery but have not been received by the facility is reduced by one and the number 306 of trays required for the surgery and received by the facility but not yet sent through the sterilizer and ready for surgery is increased by one.

In some embodiments, each number 304, 306, 308 is associated with a color. For example, the number 304 of trays required for a surgery but not been received by the facility are associated with the color blue or red, depending on the time of delivery in comparison to delivery policies set forth by the facility. In the exemplary embodiment, this number 304 appears in the first number column and is appropriately color-coded, such as in a red circle, since the delivery is in violation of the facility's policy.

The number 306 of trays required for a surgery and received by the facility but not yet sent through the sterilizer and ready for surgery are associated with the color yellow. In the exemplary embodiment, this number 306 appears in the middle number column and is appropriately color-coded, such as in a yellow circle.

The number 308 of trays required for a surgery and that have been through the sterilizer and are ready for surgery are associated with the color green. In the exemplary embodiment, this number 308 appears in the third number column and is appropriately color-coded, such as in a green circle.

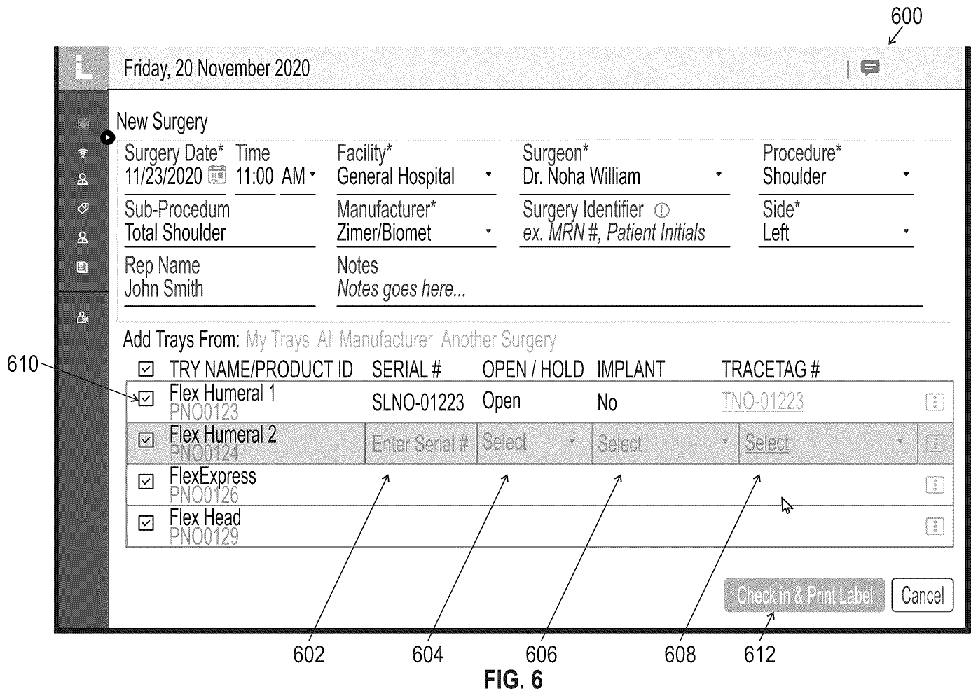

When a tray is checked in at the facility, an individual, such as the representative, physically affixes a tag to the tray (for example, placing the tag into the tray). The tag is also linked to the tray in the database, as further described in FIG. 6. Checking in a tray or trays at the facility and/or physically affixing a tag to a tray indicates that the tray has been sourced for an upcoming surgery. Once the tray is checked in at the facility, the number 304 is reduced by one and the number 306 is increased by one.

When a tray is sent through the sterilizer, the autoclave/sterilizer reaches a specified temperature for a specified period of time. The tag includes one or more sensors to determine that the specified temperature was reached, indicating that the tray has been through the autoclave. In some embodiments, after being sent through the sterilizer, the tray is wrapped to maintain the sterilization.

In some embodiments, once the tag goes into the autoclave and detects that the specified temperature was reached, the tag reports adequate sterilization to a designated beacon. The beacon transmits a notification regarding the adequate sterilization to the infrastructure transceiver. The infrastructure transceiver transmits a notification regarding the adequate sterilization to a computing device (e.g., the computing device 112 in FIG. 1). The computing device communicates with a client device (e.g., the client device 113 in FIG. 1) to reflect the adequate sterilization. For example, a GUI on the client device reflects the adequate sterilization by reducing the number 306 by one and increasing the number 308 by one.

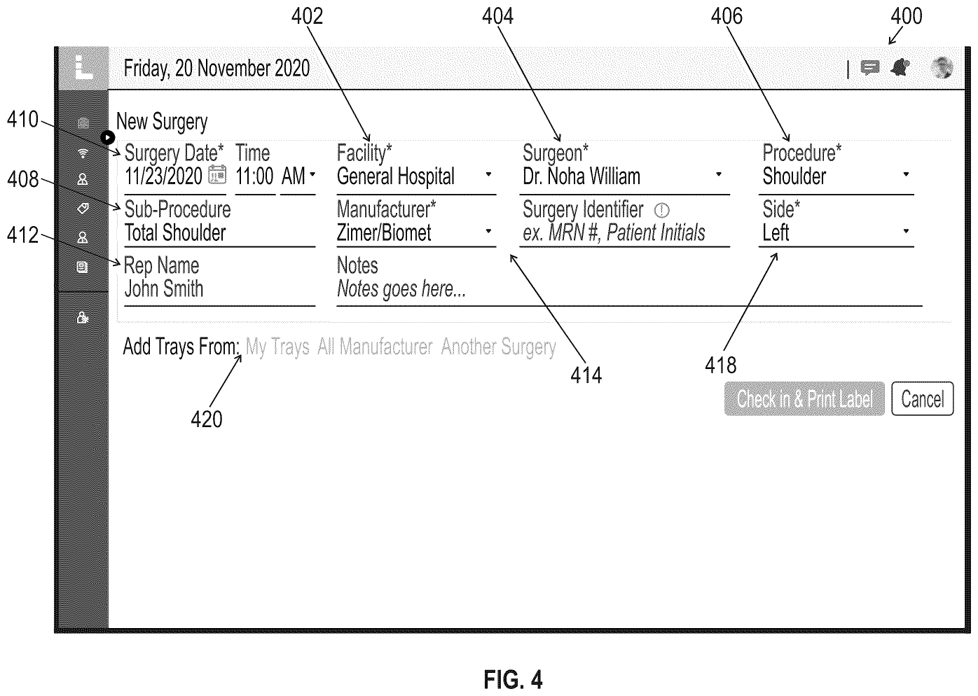

A representative may add a new surgery to interface 300 using button 318, as illustrated in FIG. 4.

FIG. 4 illustrates an exemplary dashboard user interface 400 for generating a new surgery, according to an exemplary embodiment. In some embodiments, the dashboard user interface 400 is accessed by interacting with button 318 shown in FIG. 3. In dashboard user interface 400, a user, such as a representative, may enter information associated with a new surgery, such as, but not limited to, a name of a facility where the surgery is being performed 402, a name of a surgeon 404 performing the surgery, a name of a surgery/procedure 406 and sub-surgery/procedure 408, a surgery date and time 410, a name of a representative 412, a name of a manufacturer associated with the tray(s) 414, and a side of the surgery 418 (e.g., left side or right side). In some embodiments, this information may be selectable via drop down menus. For example, in some embodiments, each drop down menu may display particular options, for example, options associated with the representative, the surgery, or the surgeon. For example, a representative may only be associated with one or more particular manufacturers or particular facilities.

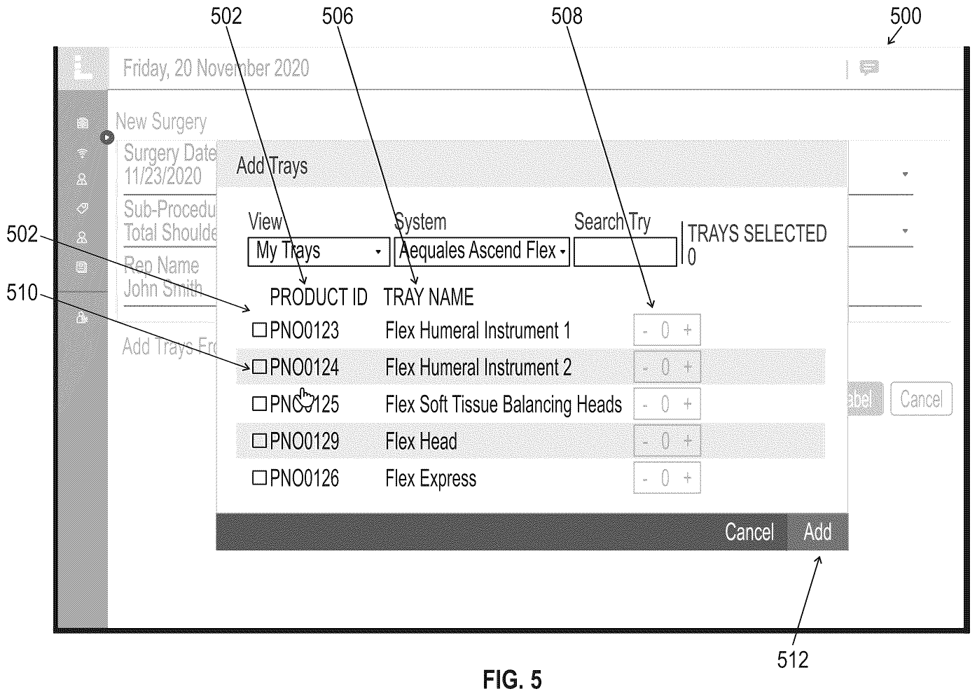

In some embodiments, the user may add trays to the surgery using button 420, as illustrated in FIG. 5.

FIG. 5 illustrates an exemplary dashboard user interface 500 for adding trays to a surgery, such as the surgery identified in FIG. 4, according to an exemplary embodiment. Trays may be added to new surgeries and/or existing surgeries. For example, a representative can use interface 500 to enter one or more trays needed for a surgery, as described below. For example, when a surgery is booked, the representative identifies the trays needed to support the surgery and uses the interface shown in FIG. 5 to enter the trays.

In some embodiments, the dashboard user interface 500 is accessed by interacting with button 420 shown in FIG. 4. In some embodiments, the trays 502 displayed in dashboard user interface 500 are entered by the user, such as the representative. For example, in some embodiments, the user types product identifiers and names of the trays into dashboard user interface 500. In other embodiments, the trays 502 displayed in the dashboard user interface 500 are automatically displayed in dashboard user interface 500 based on trays associated with the user, for example, commonly used trays by the user. In another embodiment, the trays 502 displayed in dashboard user interface 500 may be trays 502 commonly associated with the surgery and/or the surgeon. In another embodiment, the trays 502 displayed in dashboard user interface 500 may be trays 502 specific to the identified manufacturer.

In other embodiments, the trays 502 displayed in dashboard user interface 500 are pulled from a database storing tray information. For example, specific trays can be pulled from the database and displayed in dashboard user interface 500 based on particular factors, such as a procedure or a surgeon. In some embodiments, a user can create a new tray for dashboard user interface 500.

Each tray 502 displayed in the dashboard user interface 500 is associated with a selectable box 510, a product identifier 504, a tray name 506 identifying products in the tray, and a selectable number 508 of trays needed for a surgery. The user selects boxes 510 for the trays that the user wants to associate with the surgery. The user may select one or more trays via the selectable boxes 510, and add the selected trays to the surgery using button 512.

Once the new surgery is created, and optionally the trays are added to the new surgery, a new entry is created in the pre-operative dashboard user interface 300. The new entry displays the number of trays needed to be sourced for the surgery.

FIG. 6 illustrates an exemplary dashboard user interface 600 for checking in trays for a surgery, according to an exemplary embodiment. The dashboard user interface 600 illustrates checking in the trays added in FIG. 5. In the exemplary embodiment, the user may enter and/or select a serial number 602 associated with a tray, whether the tray may be immediately opened once it arrives in the surgery room or whether the surgery room should hold off on opening the tray 604 (for example, hold indicates not to immediately unwrap the tray as the tray may not be needed for surgery), whether the tray includes an implant 606, and a tag identifier associated with the tray 608. In some embodiments, a default answer based on what the tray contains is added for whether the tray includes an implant 606. In some embodiments, this information may be selectable via drop down menus. For example, in some embodiments, the user selects a tag by using a drop down menu to view the tags within the facility and selecting a tag to associate with the tray.

In some embodiments, this information may be added via scanning technologies. For example, in some embodiments, the user identifies the trace tag by scanning a barcode on the tag. The user may select one or more trays via selectable boxes 610 associated with the one or more trays. The user may check in and print labels for the selected trays using button 612. In some embodiments, a user can select an option in interface 600 to identify that one or more instruments are missing from a tray.

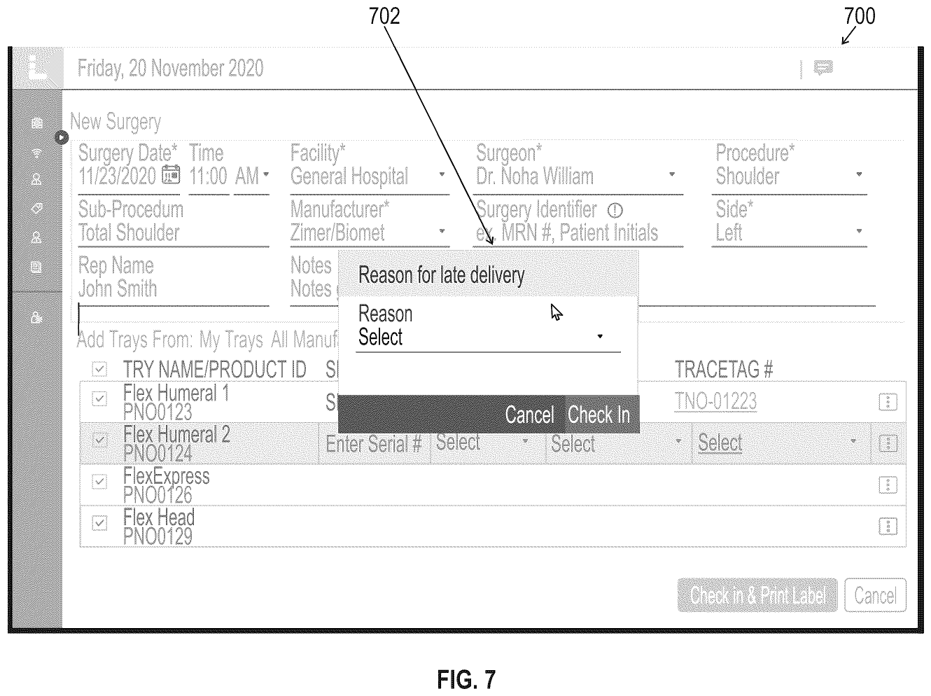

FIG. 7 illustrates an exemplary dashboard user interface 700 with a pop-up window 702 for entering a reason for a late delivery of trays, according to an exemplary embodiment. For example, facilities may have policies that trays are to be sourced within a specified amount of time before a surgery (for example, 24 hours or 48 hours before a surgery). If the trays are checked in for a surgery past the policy deadline, a user may enter a reason for a late delivery in window 702.

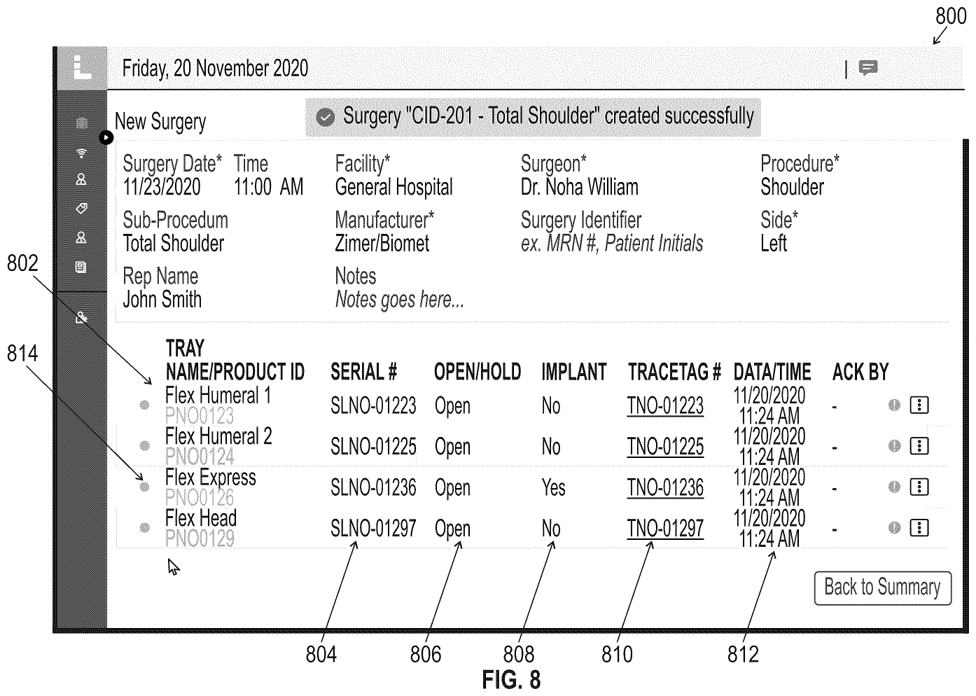

FIG. 8 illustrates an exemplary dashboard user interface 800 displaying trays 802 checked in for a surgery, according to an exemplary embodiment. In the exemplary embodiment, dashboard user interface 800 includes the serial number 804 associated with the tray, whether the tray may be immediately opened once it arrives in the surgery room or whether the surgery room should hold off on opening the tray 806, whether the tray includes an implant 808, the tag identifier, represented as a hyperlink, associated with the tray 810, and a date and time that the trays were checked in at the facility 812.

In one embodiment, each tray 802 is associated with a color-code indicator 814. For example, as illustrated, the trays 802 are associated with yellow circle indicators 814 indicating that the trays have been checked in at the facility but have not yet been sent through the sterilizer.

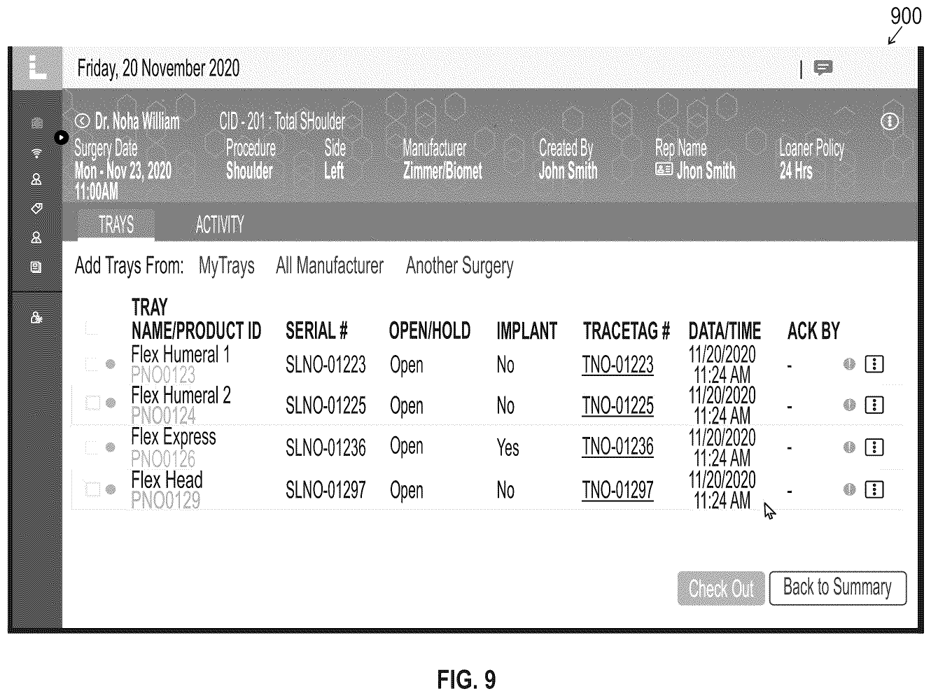

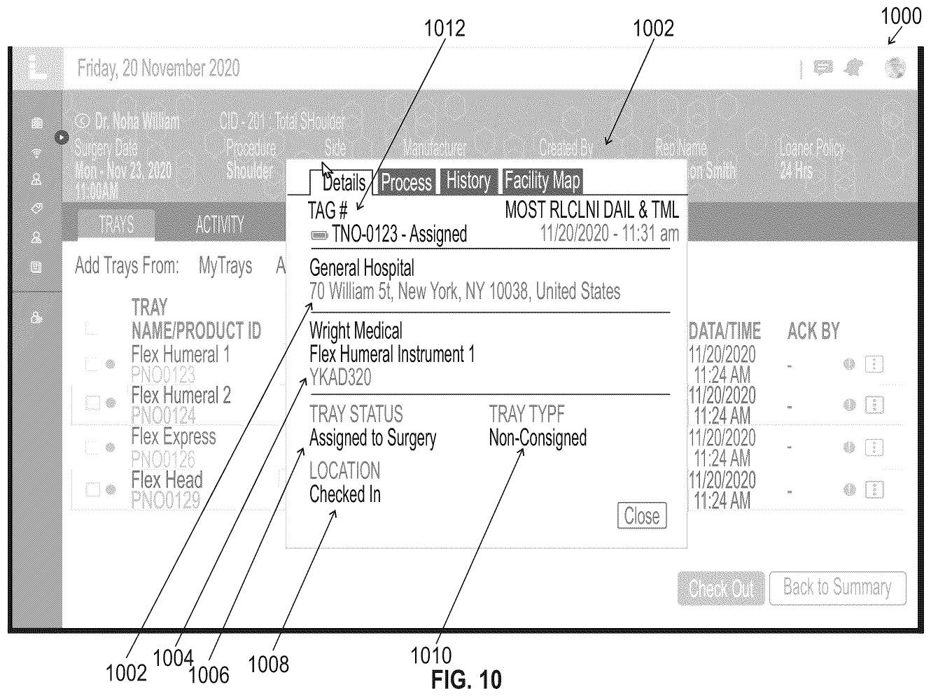

FIG. 9 illustrates an exemplary dashboard user interface 900 displaying a summary of existing surgeries and the trays displayed in FIG. 8, according to an exemplary embodiment. A user may select or click a tag identifier to view further information, as illustrated in FIGS. 10-13.

FIG. 10 illustrates an exemplary dashboard user interface 1000 displaying a window 1002 displaying information associated with a tag selected in FIG. 9, according to an exemplary embodiment. In the illustrated embodiments, the dashboard user interface 1000 displays a name of a facility where the tray is checked in 1002, a name of the tray 1004 and/or the manufacturer associated with the tray, a surgery status of the tray 1006, a location of the tray 1008, a type of tray 1010 (for example, consigned, non-consigned, or turn over), and a tag identifier assigned to the tray 1012.

In an exemplary embodiments, the location of the tray 1008 is based on the location of the tag assigned to the tray.

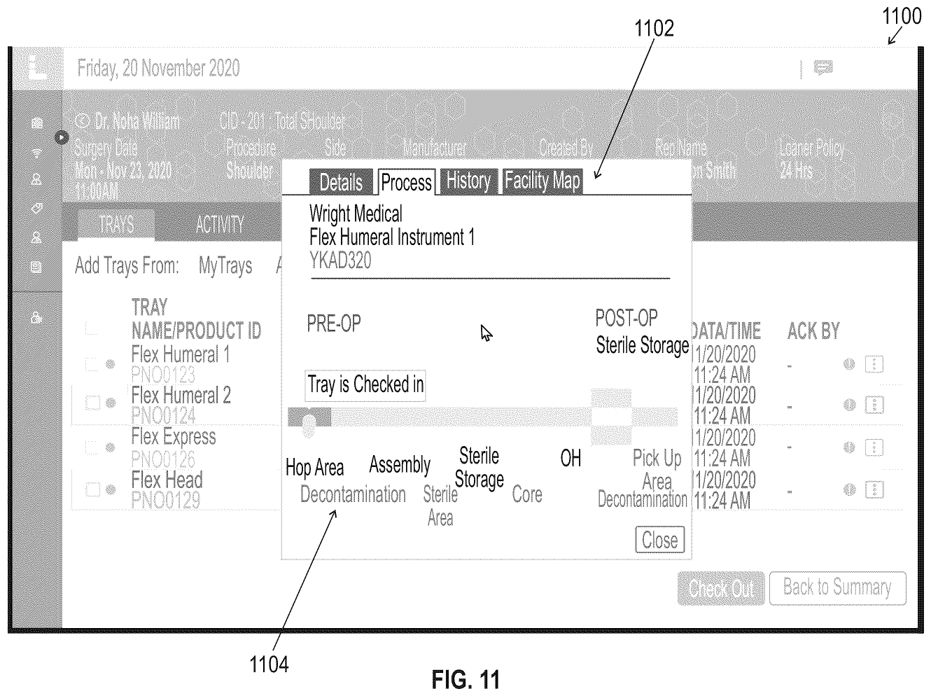

FIG. 11 illustrates an exemplary dashboard user interface 1100 displaying a progress window 1102 associated with the tray selected in FIG. 9, according to an exemplary embodiment. The progress window 1102 includes a progress bar 1104 that displays steps associated with the tray from the time of being checked-in to the facility until post-surgery. The progress bar 1104 is updated based on the location and temperature information gathered from a tag communicating with beacons located in different areas.

In the illustrated embodiment, the pre-operative steps or locations are representative area, decontamination, assembly, sterile area, sterile storage, care, and operating room (OR). In the illustrated embodiment, the post-operative steps are decontamination, sterile storage, and pick-up area. However, in other embodiments, there may be different steps or locations. For example, in some embodiments, the steps or locations are facility configured. In some embodiments, each step or location is associated with a particular area or room.

In some embodiments, each step or location is associated a beacon for communicating with a tag and automatically updating the progress bar. For example, when a tag associated with a tray enters the OR, the tag communicates with a beacon located in or near the OR. As described herein, a computing device (e.g., IoT computing device 112) receives the location information and updates a GUI (e.g., GUI 124) on a user computing device (e.g., computing device 113) based on the location.

As described further in FIGS. 1 and 2, one or more rooms or areas in the facility include one or more beacons used to track the tag. The beacons can correspond to one or more areas within the facility. The beacons can be implemented utilizing a near field communication technology including but not limited to Bluetooth LE. The beacon can be connected to the infrastructure transceiver utilizing the same communication technology for tag detection or a different communication technology. For example, the beacons can interface with the tag utilizing Bluetooth LE and additionally connect with the infrastructure transceiver utilizing a WiFi connection. In some embodiments, the tag can be a near field communication device that operates in a low power envelope. As the tag comes within the broadcast range of the beacon, the tag and the beacon present a handshake. The beacon identifies the tag and maintains a connection with the tag utilizing periodic polling. The beacon communicates with the infrastructure transceiver notifying the computing device through the network as to which beacon the tag is currently associated. The computing device correlates the location of the beacon with an area within a facility and determines that the tag is located that particular area. The computing device updates the progress bar 1104 based on the area that the tag is located. The steps can move forward or backwards within the progress bar 1104 based on the location of the tag.

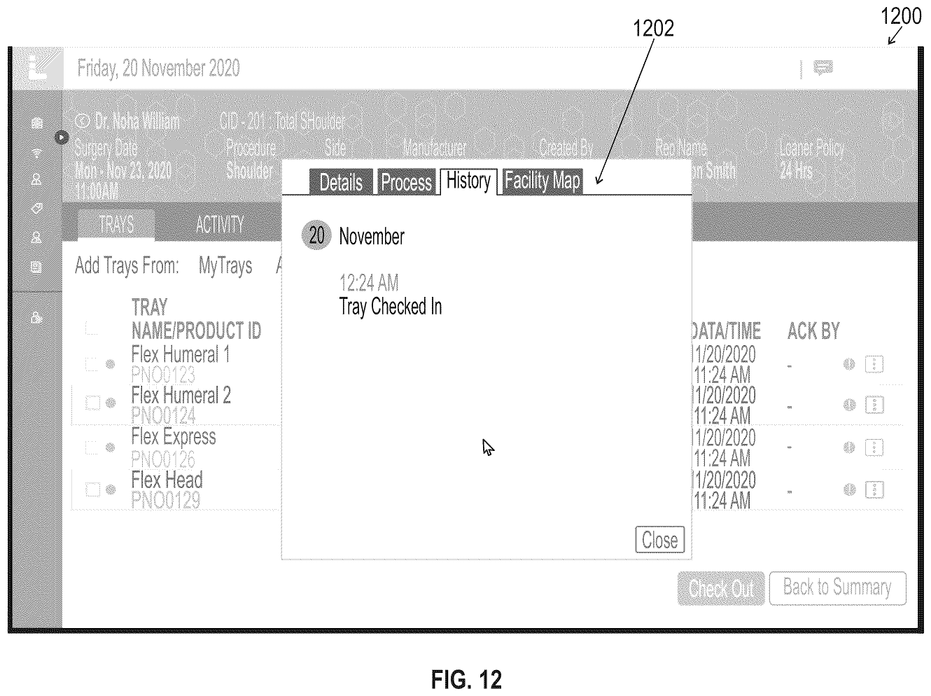

FIG. 12 illustrates an exemplary dashboard user interface 1200 displaying a history window 1202 associated with the tray selected in FIG. 9, according to an exemplary embodiment. In some embodiments, the history window 1202 displays a location history associated with the tray, with date and time stamps.

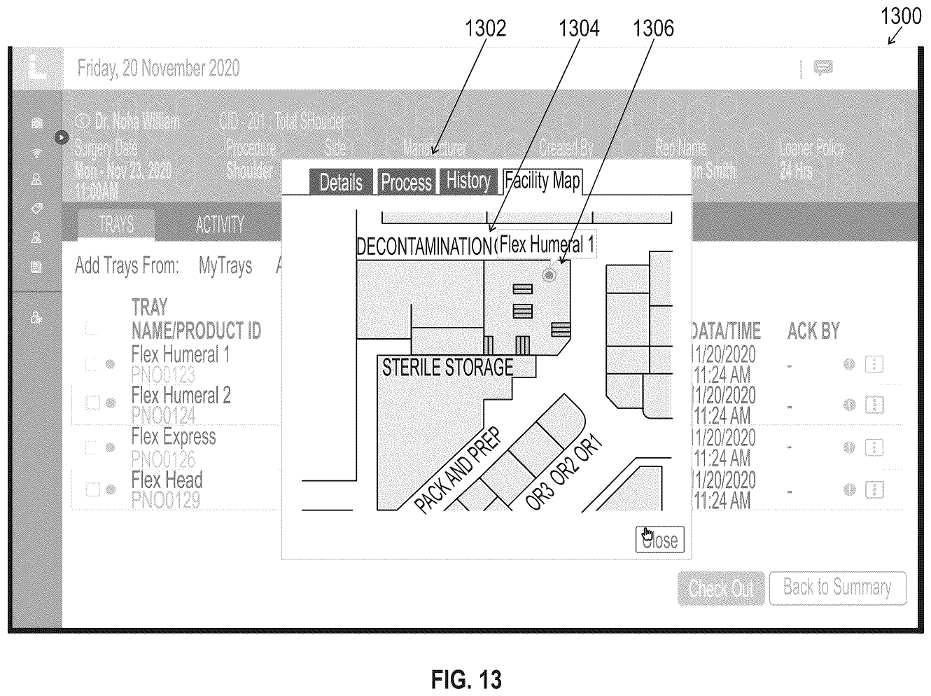

FIG. 13 illustrates an exemplary dashboard user interface 1300 displaying a mapping window 1302 associated with the tray selected in FIG. 9, according to an exemplary embodiment. The mapping window 1302 displays a facility map 1304 displaying a location 1306 of the tray on the facility map. The location of the tray is based on the tag associated with the tray communicating with one or more beacons located in the area The facility map 1304 is facility dependent and changes based on the layout of the facility.

While FIGS. 10-13 are illustrated as pop-up windows, in other embodiments, these windows may be displayed as a whole screen (e.g., a webpage or via an application).

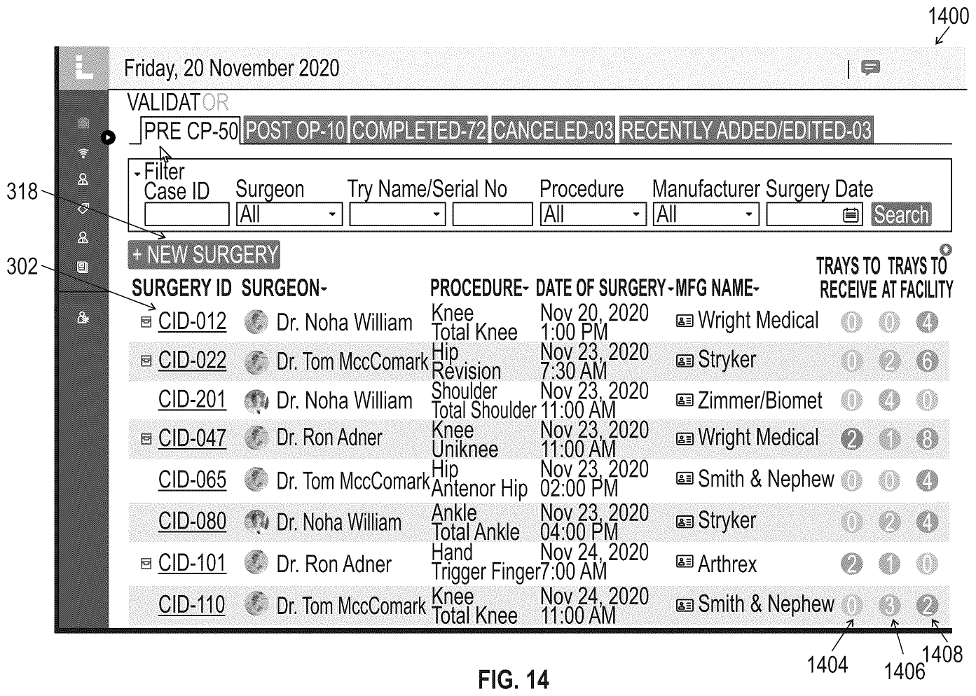

FIG. 14 illustrates an exemplary pre-operative dashboard user interface 1400 displaying a list of surgeries that required one or more surgical tray, according to an exemplary embodiment. The pre-operative dashboard user interface 1400 further includes a number 1404 of trays required by the surgery but have not been received by the facility. In the exemplary embodiment, this number 1404 appears appropriately color-coded, such as in a red circle.

The pre-operative dashboard user interface 1400 further includes a number 1406 of trays that have been received by the facility but that have not been through the sterilizer. In the exemplary embodiment, this number 1406 appears appropriately color-coded, such as in a yellow circle.

The pre-operative dashboard user interface 1400 further includes a number 1408 of trays that have been received by the facility, that have been through the sterilizer, and are ready for surgery. In the exemplary embodiment, this number 1408 appears appropriately color-coded, such as in a green circle.

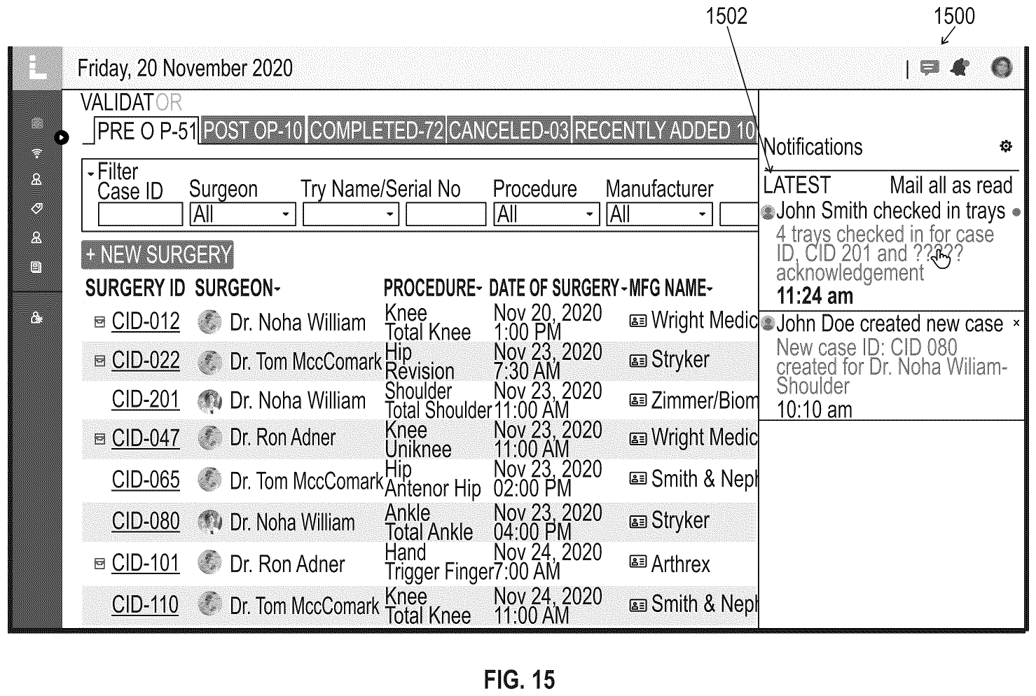

FIG. 15 illustrates an exemplary pre-operative dashboard user interface 1500 illustrating notifications transmitted to one or more parties based on events and/or based on actions performed in the described dashboards, according to an exemplary embodiment. For example, these notifications can be driven by actions taken (or not taken) by users as well as IoT driven information, which would mean they are system generated.

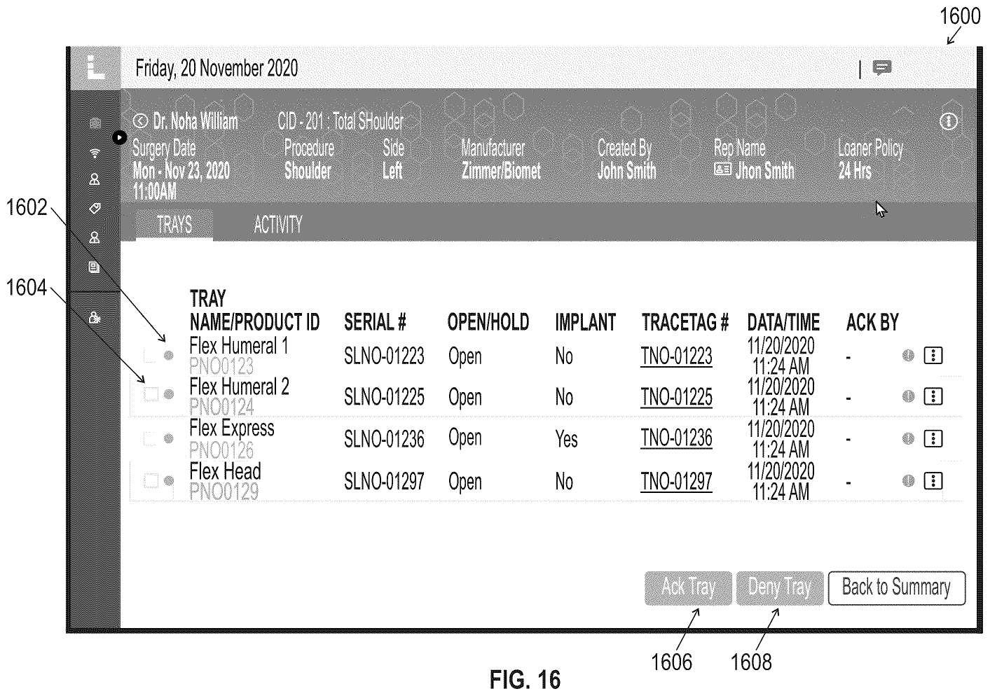

FIG. 16 illustrates an exemplary pre-operative dashboard user interface 1600 in which an administer for the facility can acknowledge or deny receiving trays for a surgery after these trays are checked in by a manufacturer representative, according to an exemplary embodiment. The dashboard user interface 1600 displays one or more trays 1602 associated with a surgery that have recently been checked in by a representative, for example, using the dashboard user interface 600 for adding trays identified in FIG. 6. The administer may select one or more trays via selectable boxes 1604 associated with the trays, and acknowledge or deny the selected trays using the acknowledge button 1606 or the deny button 1608.

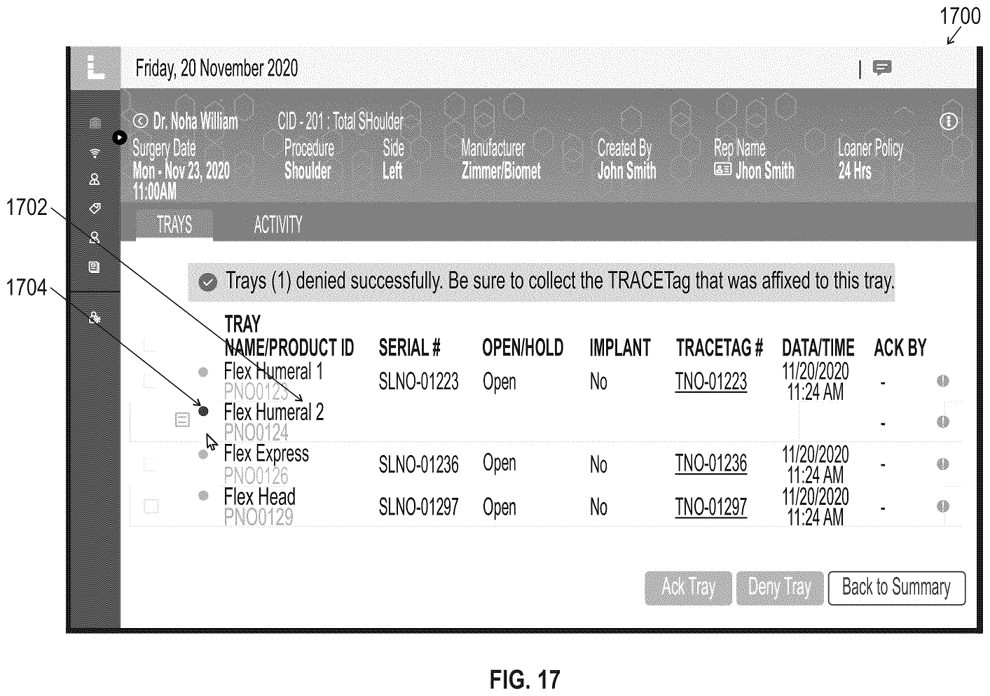

FIG. 17 illustrates an exemplary pre-operative dashboard user interface 1600 in which an administer for the facility has denied receiving a tray 1702 for a surgery using the dashboard user interface 1600 shown in FIG. 16, according to an exemplary embodiment. Denying a tray prevents the tray from being checked in at the facility and going through processing. In some embodiments, a notification is sent to the representative to retrieve the tray or deal with an issue causing the tray to be denied. In some embodiments, as a result of denying the tray, a color coded identifier 1704 associated with the denied tray changes colors, for example, from yellow to red or blue.

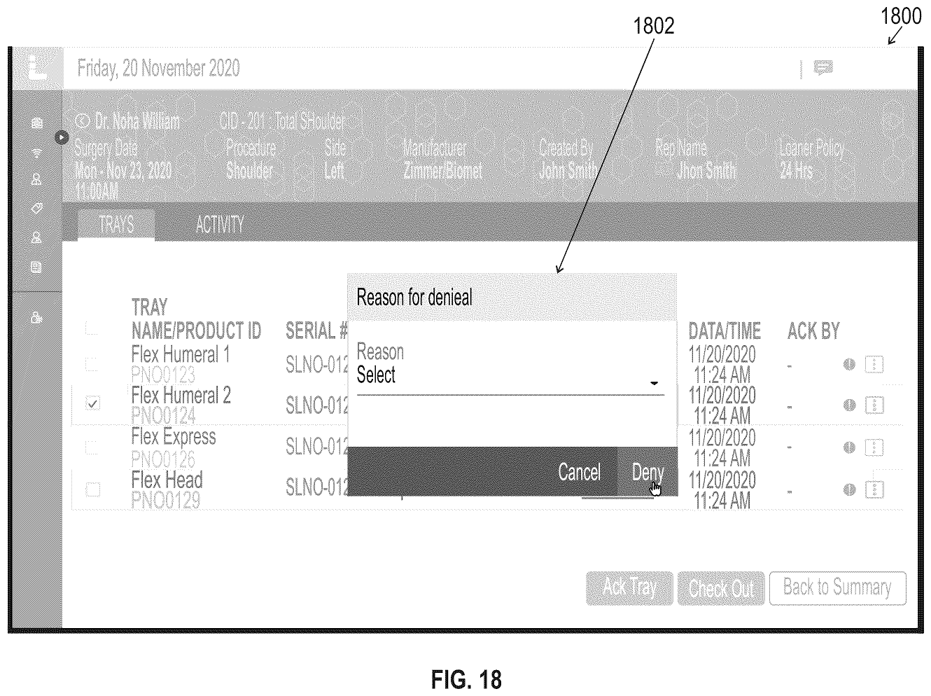

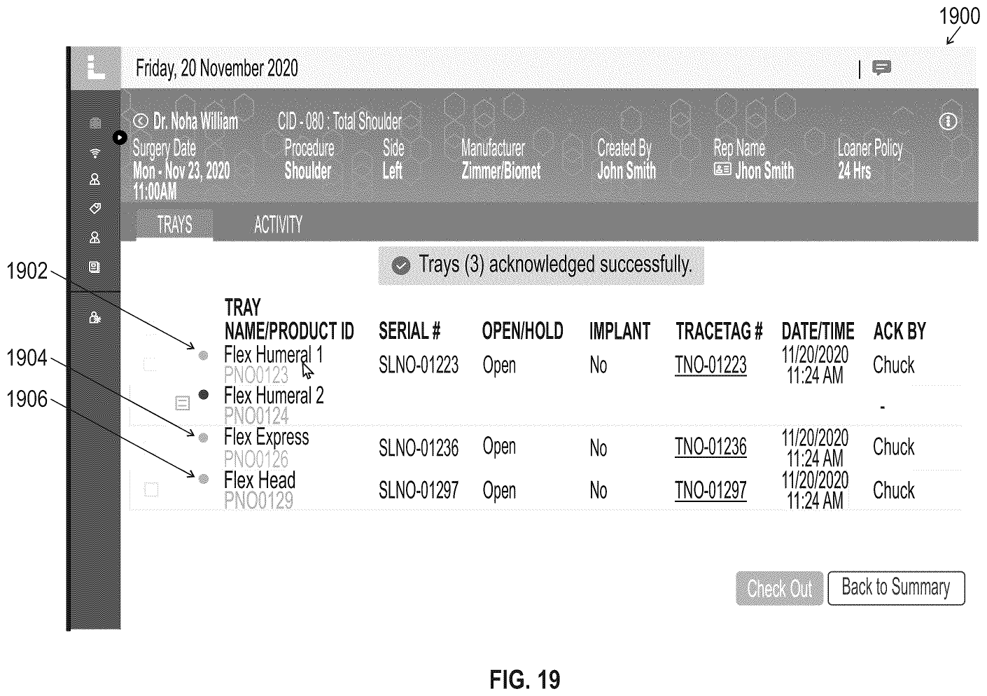

FIG. 18 illustrates an exemplary pre-operative dashboard user interface 1800 displaying a window 1802 in which the administer for the facility can identify a reason for denying the tray for the surgery as shown in FIG. 17, according to an exemplary embodiment.

FIG. 19 illustrates an exemplary pre-operative dashboard user interface 1900 in which an administer for the facility has acknowledged receiving trays 1902, 1904, 1906 for a surgery using the dashboard user interface 1606 shown in FIG. 16, according to an exemplary embodiment.

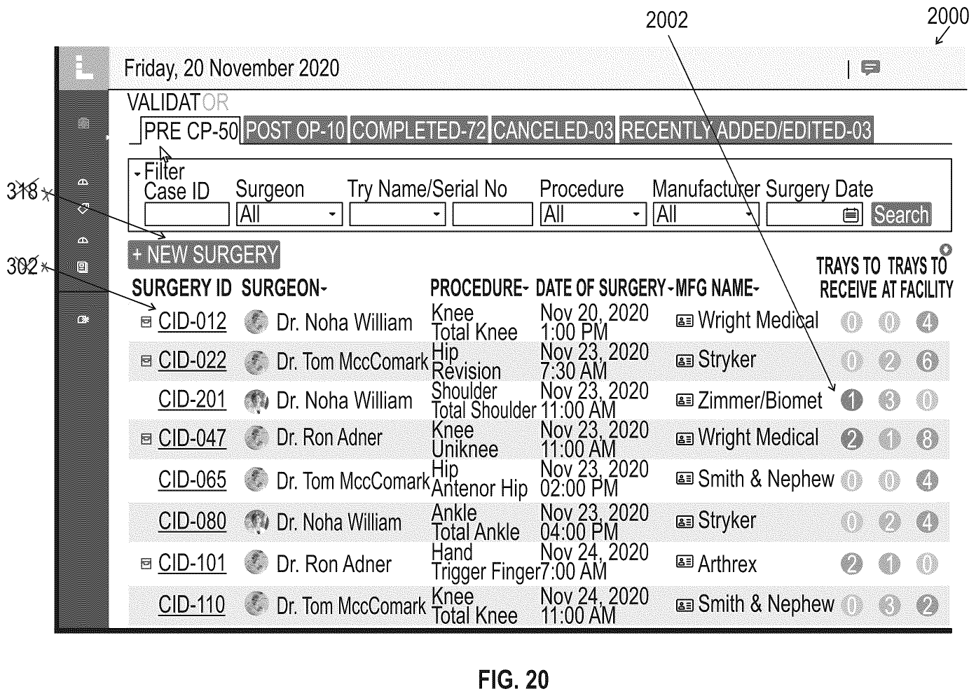

FIG. 20 illustrates an exemplary pre-operative dashboard user interface 2000 corresponding to the pre-operative dashboard user interface 1400 of FIG. 14 updated to reflect that one tray for the surgery needs to be re-delivered because the tray was denied, as shown in FIG. 17, according to an exemplary embodiment. As a result of the tray denial, the pre-operative dashboard user interface 2000 includes an increased number 2002 of trays required by the surgery but have not been received by the facility (here, one tray was denied, therefore the number 2002 of trays required by the surgery but have not been received by the facility increases by one). In the exemplary embodiment, this number 2002 appears appropriately color-coded, such as in a red circle. As a result of the tray denial, a tray will need to be re-sourced (e.g., consigned or a turn over) or redelivered.

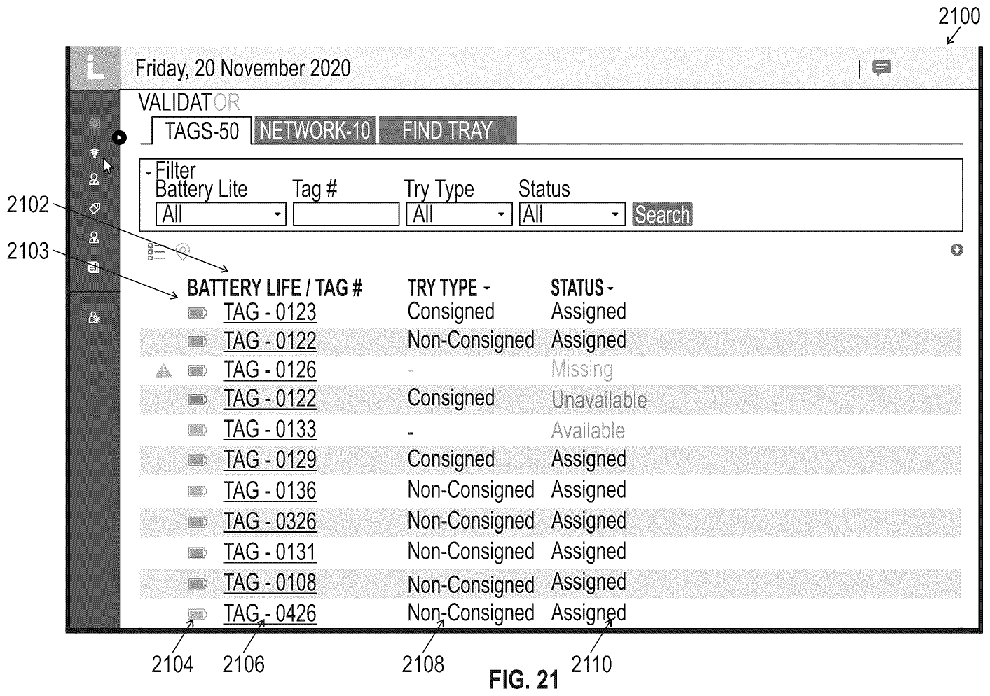

FIG. 21 illustrates an exemplary dashboard user interface 2100 displaying a list of tags 2102, according to an exemplary embodiment. For each tag 2103, the list 2102 includes a battery life of the tag 2104, a tag identifier 2106, a type of tray the tag is associated with (e.g. either non-consigned or consigned) 2108, and a status of the tag 2110 (e.g., assigned, missing, unavailable, or available).

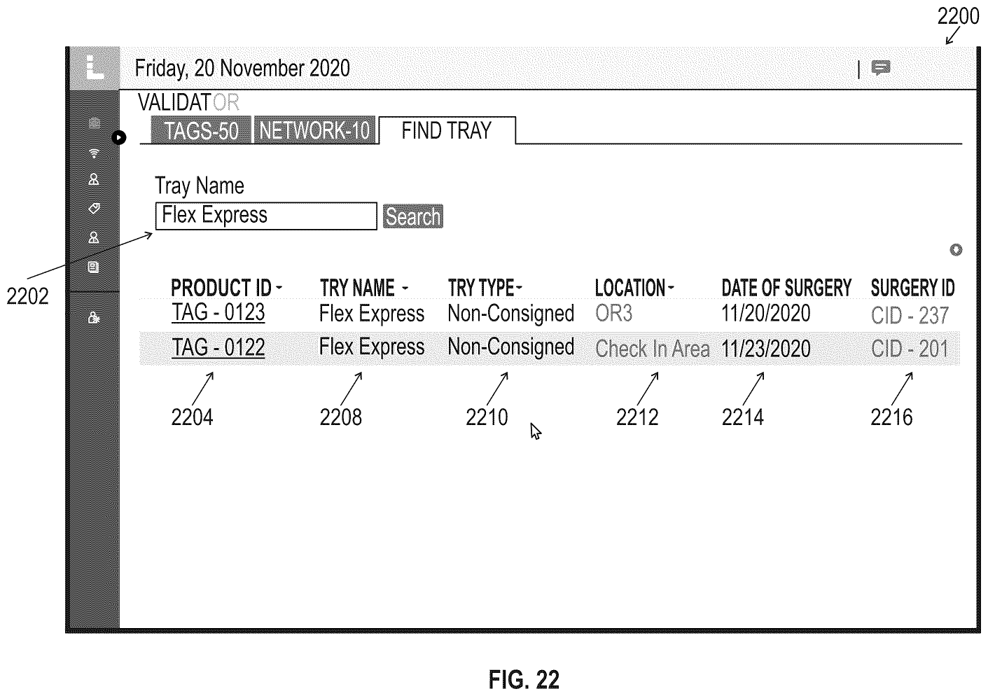

FIG. 22 illustrates an exemplary dashboard user interface 2200 displaying tray information, according to an exemplary embodiment. In the embodiment, a user is searching a tray name 2202 (“Flex Express”). The results of the search displays “Flex Express” trays that are checked in and associated with tags. The interface 2200 displays for each result a product identifier 2204, a tray name 2208, a tray type 2210 (e.g., whether the tray is consigned), a location of the tray 2212 (provided through IoT information), a date of surgery 2214, and a surgery identifier 2216.

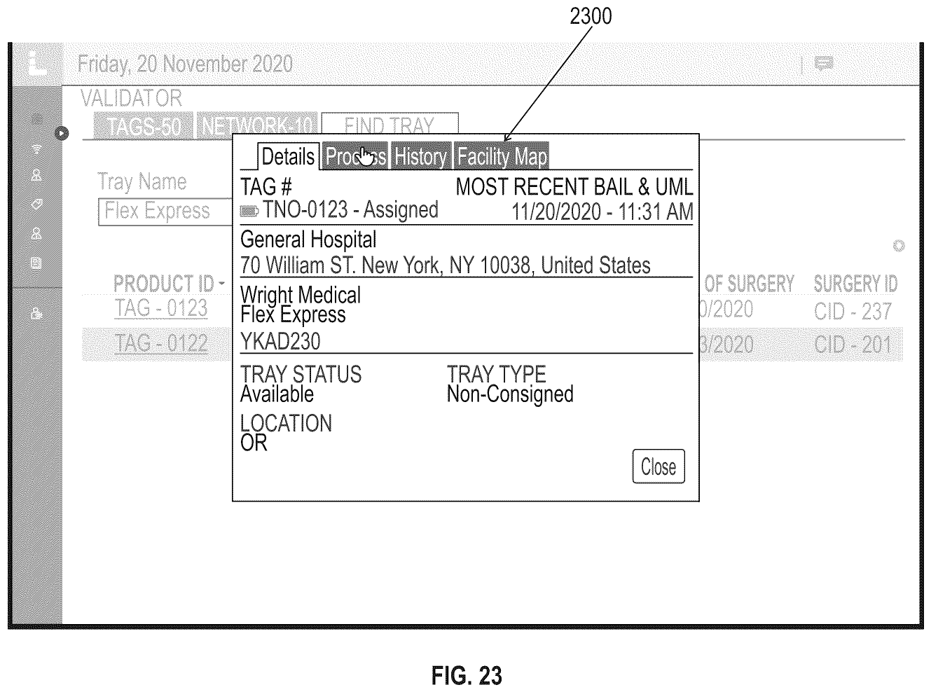

FIG. 23 illustrates an exemplary window 2300 displaying details associated with a tag associated with a tray, according to an exemplary embodiment. For example, a user may select a tag shown in FIG. 22 to view window 2300 for the tag. In an exemplary embodiment, window 2300 displays a tag identifier for the tag, a name of a hospital where the tag is located, a name of a medical manufacturer associated with the tray, a tray status, a location of the tray based on a location of the tag, a tray type, and/or a battery charge of the tag.

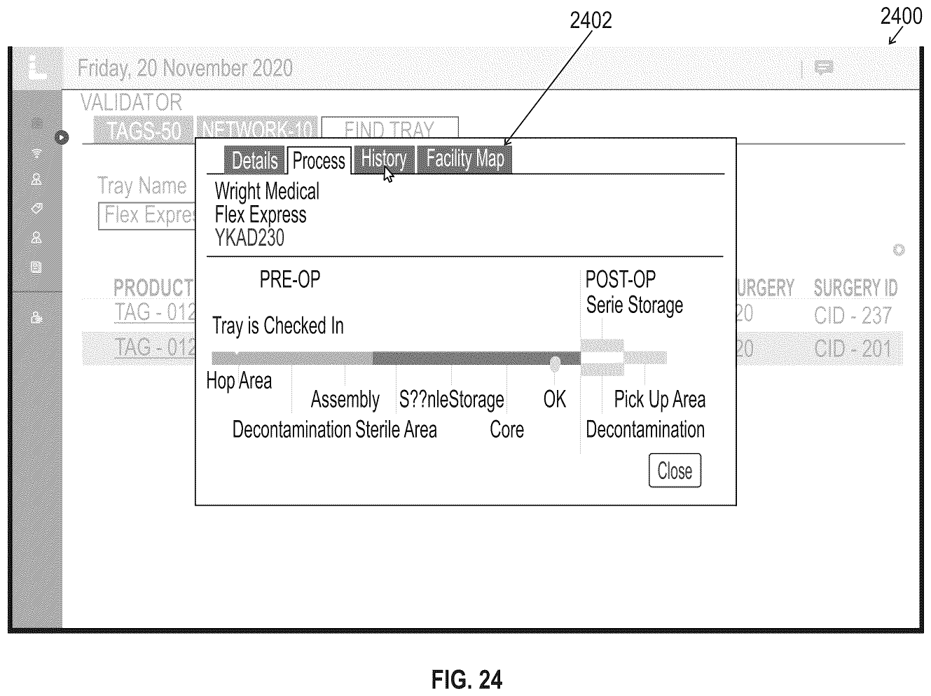

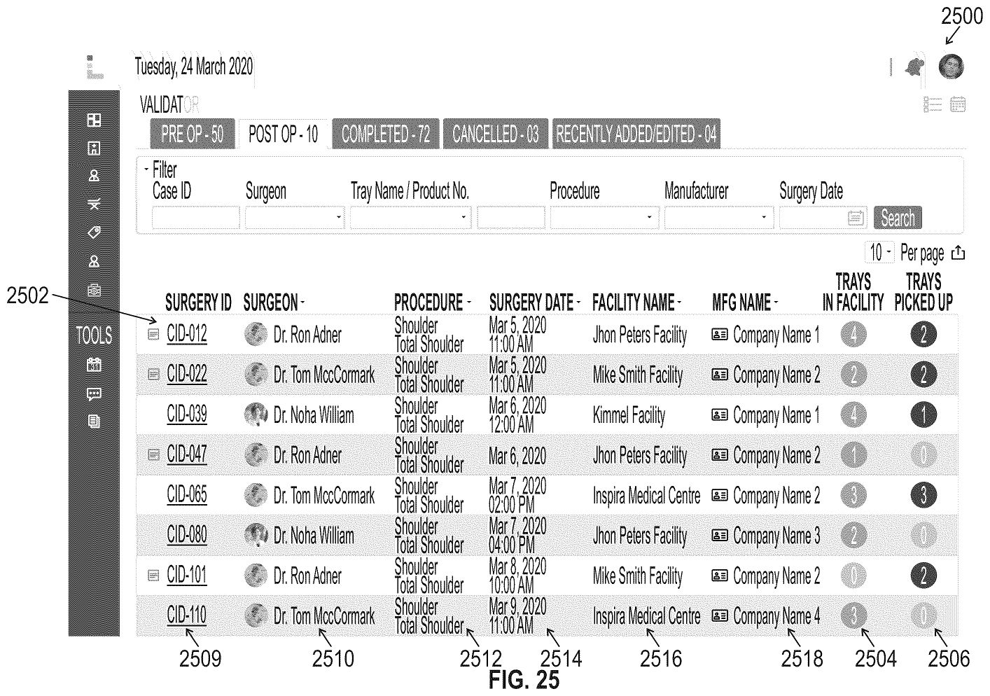

FIG. 24 illustrates an exemplary window 2400 displaying a progress bar 2402 for a tag shown in FIG. 22, according to an exemplary embodiment. The progress bar 2402 displays each step of the process and where the tag is located in the process.

FIG. 25 illustrates an exemplary post-operative dashboard user interface 2500 illustrating post-surgery trays, according to an exemplary embodiment. Each entry 2502 listed in the interface 2500 is associated with one or more trays. Each entry 2502 listed in the interface 2500 includes a number 2504 of trays in the facility but have not been retrieved from the facility or are unassigned from surgeries. Each entry 2502 listed in the interface 2500 also includes a number 2506 of trays that have been retrieved from the facility or are unassigned from surgeries. The numbers are updated as a tray is retrieved from the facility. For example, as a tray is retrieved from a facility, the number 2504 of trays in the facility but have not been retrieved from the facility is reduced by one and the number 2506 of trays retrieved from the facility is increased by one.

In some embodiments, each number 2504, 2506 is associated with a color. For example, the number 2504 of trays in the facility but have not been retrieved from the facility are associated with the color blue. The number 2506 of trays retrieved from the facility are associated with the color black.

When a tray is being pulled out of a hospital, the tag is disassociated from the tray by checking the tray out in the system and physically removing the tag. The representative would check out the tray to break the link of the tag from tray.

Each entry 2502 is further associated with relevant information, such as, but not limited to, a surgery identifier 2509, a name of a surgeon performing the surgery 2510, a name of a procedure 2512, a surgery date and time 2514, a name of a facility where the surgery is being performed 2516, a name of a manufacturer associated with the tray(s) 2518, a number of trays located in facility 2504, and a number of trays retrieved from the facility 2506.

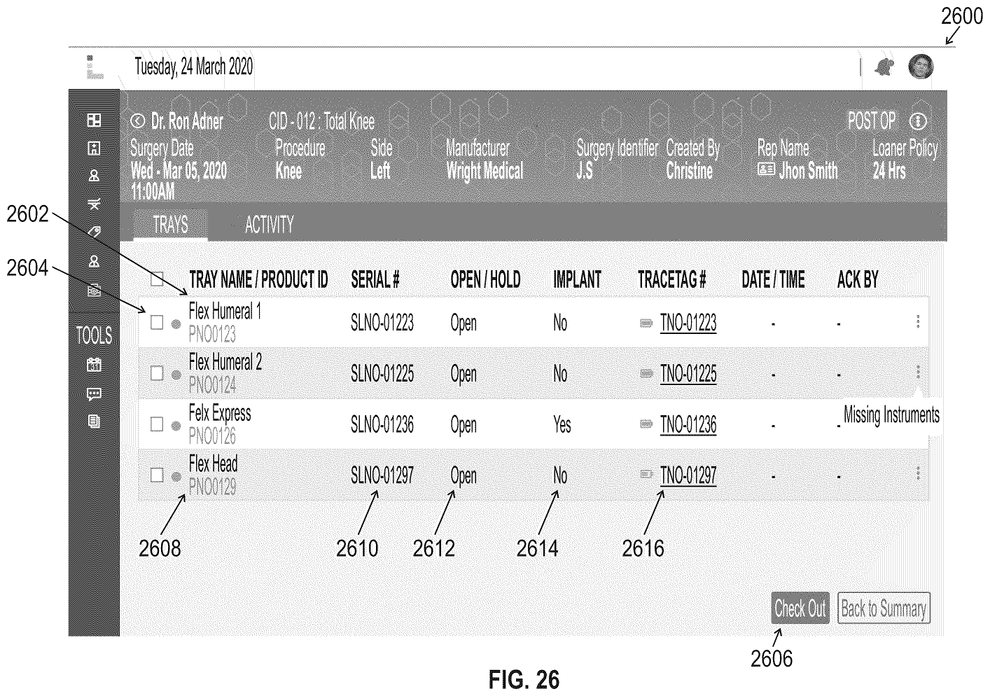

FIG. 26 illustrates an exemplary post-operative dashboard user interface 2600 displaying post-operative trays associated with a surgery performed a physician, according to an exemplary embodiment. The representative may select one or more trays via selectable boxes 2604 associated with the trays, and “checkout” the selected trays using the checkout button 2606. The representative checks out the tray to break the link of the tag from tray.

In the exemplary embodiment, dashboard user interface 2600 includes a serial number 2610 associated with the tray, a tray name 2602, tray product identifier 2608, serial number 2610 and open/hold designation 2612, indicating whether the tray could be immediately opened once it arrives in the surgery room or whether the surgery room should hold off on opening the tray, whether the tray includes an implant 2614, and the tag identifier associated with the tray.

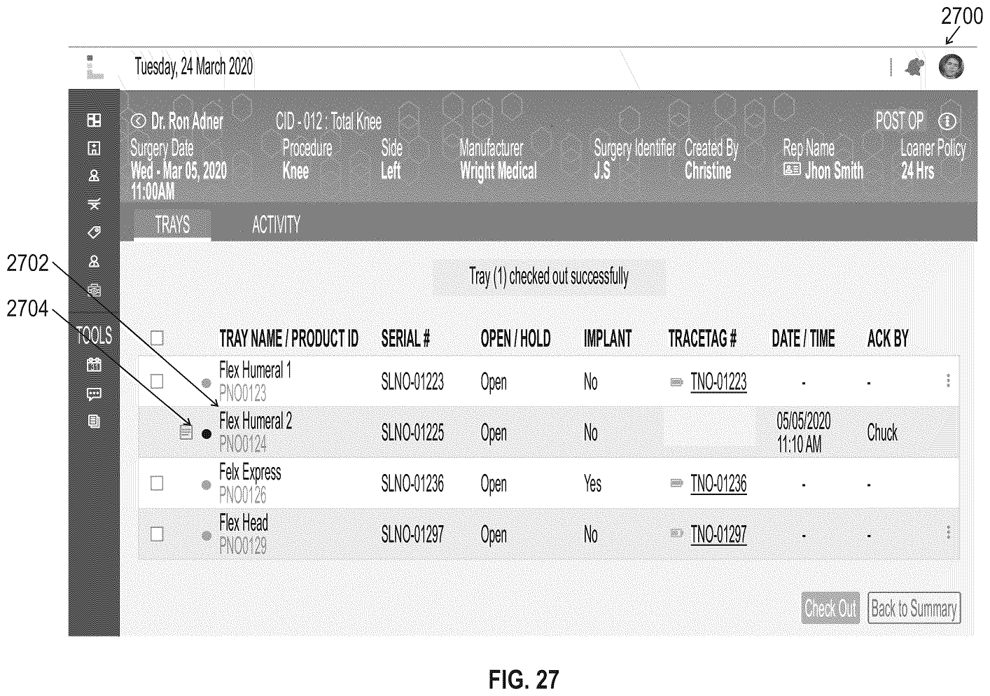

FIG. 27 illustrates an exemplary post-operative dashboard user interface 2700 displaying a tray 2702 that has been checked out using the dashboard user interface 2600 shown in FIG. 26, according to an exemplary embodiment. In some embodiments, as a result of checking out the tray, a color coded identifier 2704 associated with the tray 2702 changes colors, for example, from blue to black. When the representative checks out the tray, this breaks the link between the tag and the tray. The tag identifier is no longer a link, which brings attention to the user that the link between the tag and the tray is broken.

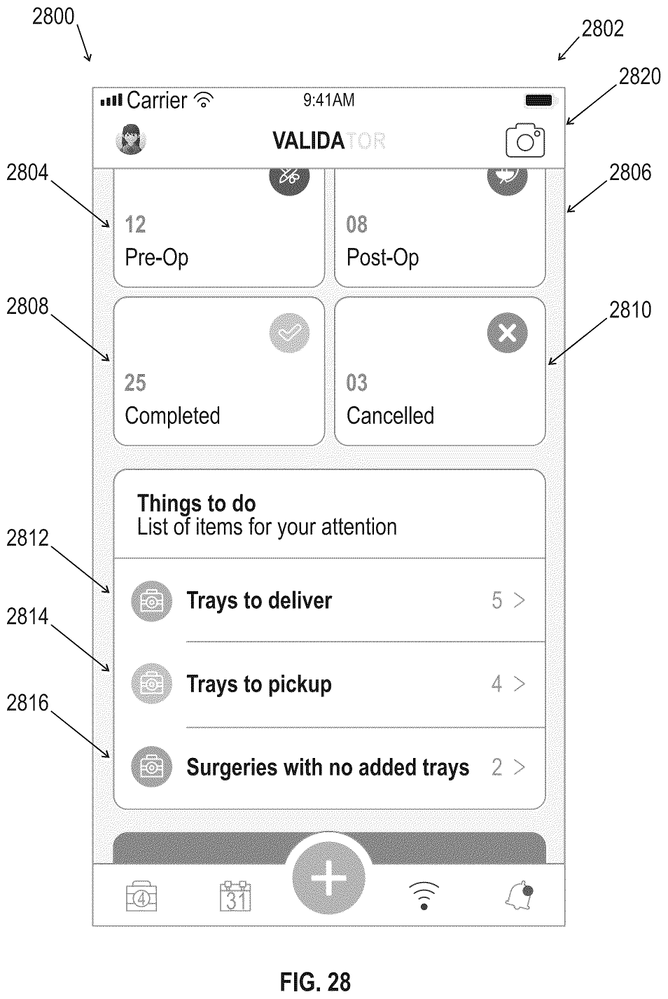

FIG. 28 illustrates an exemplary mobile application user interface 2800 displaying a dashboard user interface 2802 displaying selectable graphics associated with pre-operative actions 2804 (e.g., scheduled surgeries), post-operative actions 2806 (e.g., after surgeries), completed 2808 (e.g., completed surgeries), and cancelled 2810 (e.g., surgeries that have been canceled), according to an exemplary embodiment. Selectable graphics 2804, 2806, 2808, 2810 also represent corresponding tabs in the web design shown in FIG. 3.

The dashboard user interface 2802 further includes selectable graphics associated with trays tobe delivered for upcoming surgeries across facilities 2812, trays to retrieved across facilities 2814, and surgeries which need representatives to add trays 2816.

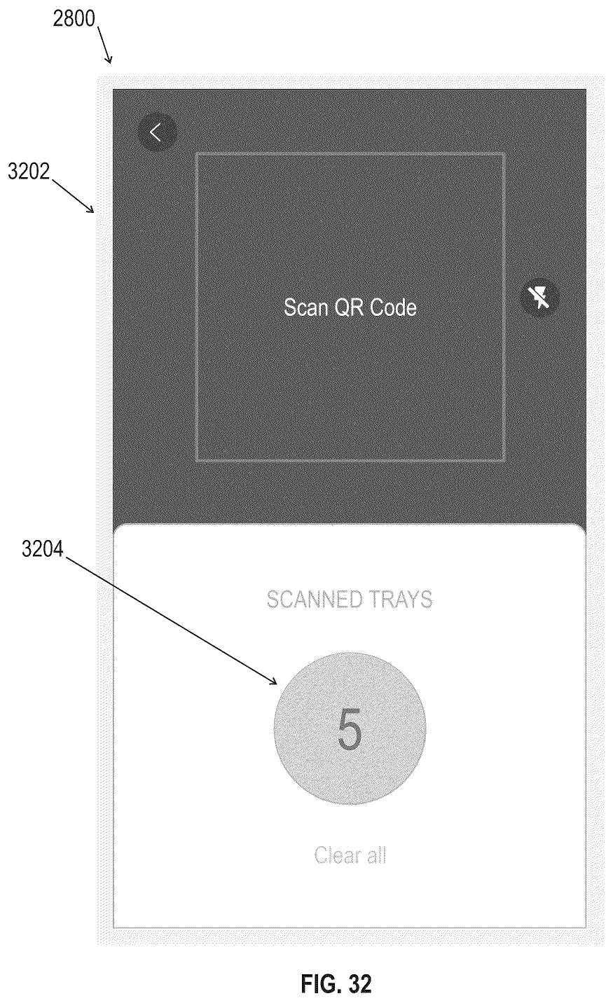

The dashboard user interface 2802 further includes a camera icon 2820 to invoke mobile phone scanning software for scanning trays, as shown in FIG. 32.

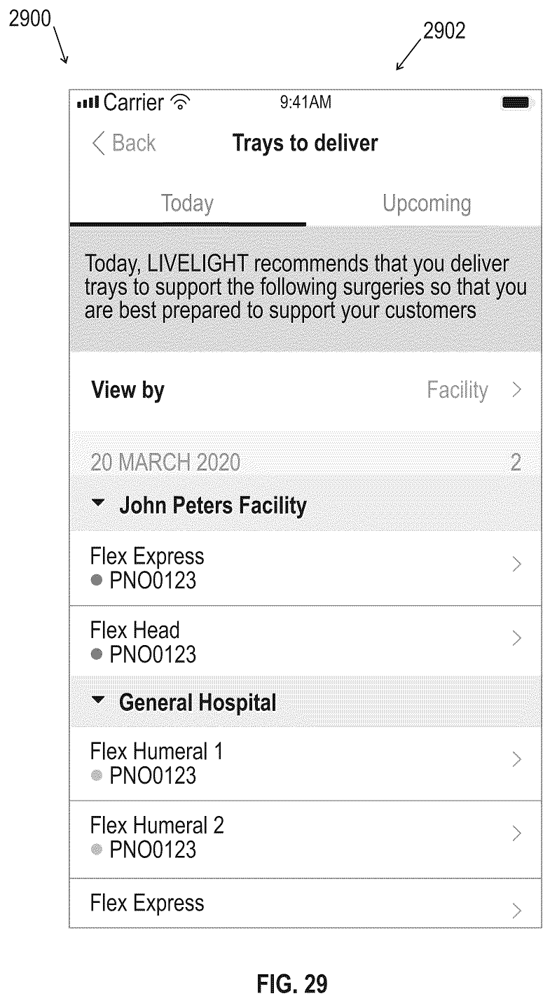

FIG. 29 illustrates an exemplary mobile application user interface 2900 displaying a dashboard user interface 2902 displaying trays to be sourced to facilities grouped by facilities, according to an exemplary embodiment. Interface 2902 displays a list of facilities. Each facility is visually associated on interface 2902 with one or more trays needed to be sourced to that facility.

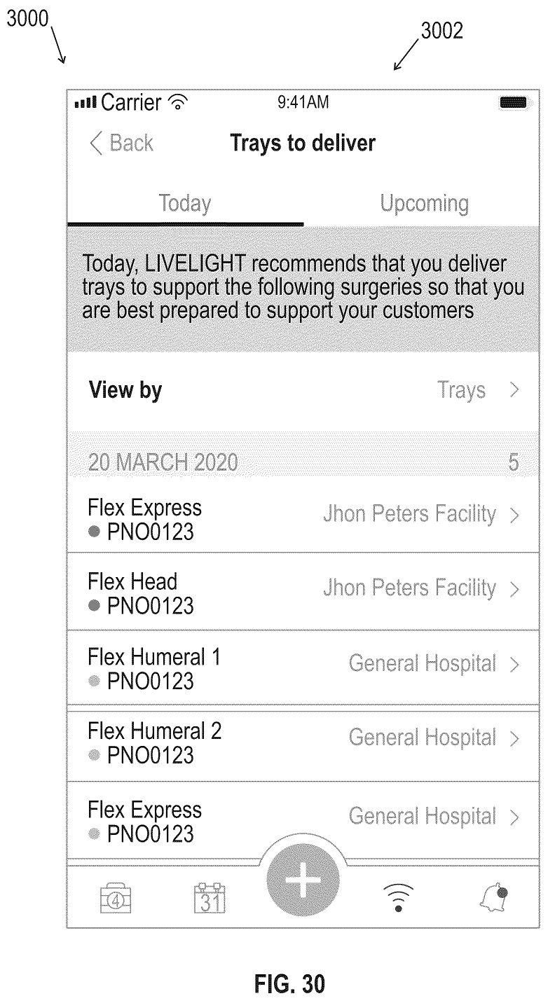

FIG. 30 illustrates an exemplary mobile application user interface 3000 displaying a dashboard user interface 3002 displaying trays to be sourced to facilities filtered based on the tray name, according to an exemplary embodiment. Interface 3002 displays a list of trays. Each tray is visually associated on interface 3002 with the name of the tray, the product ID, and a facility where that tray needs to be sourced.

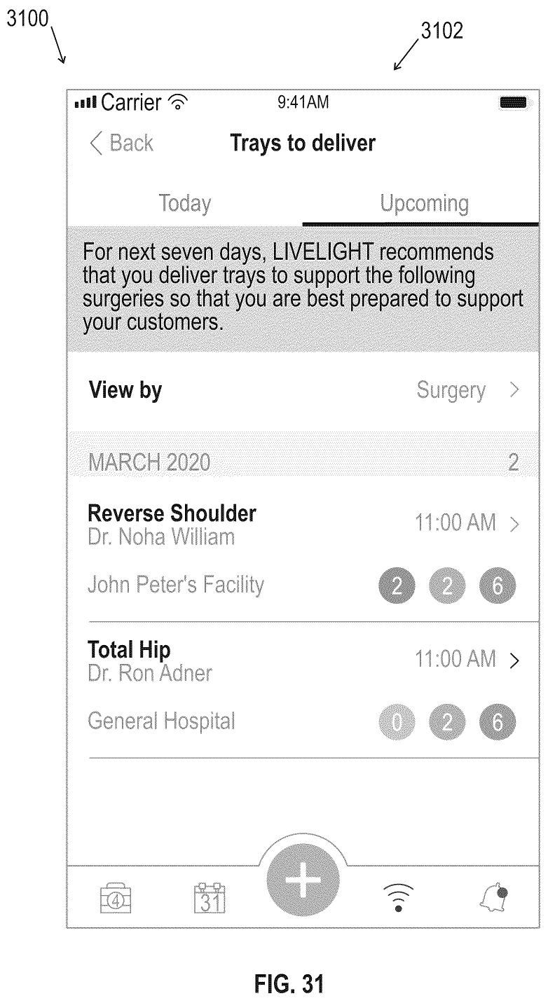

FIG. 31 illustrates an exemplary mobile application user interface 3100 displaying a dashboard user interface 3102 displaying trays to be sourced to facilities filtered based on the surgeries, according to an exemplary embodiment. Interface 3102 displays a list of surgeries. Each surgery is visually associated on interface 3102 with a name of a surgeon performing the surgery, a name of a facility where the surgery is being performed, a number of trays needed to be sourced to the facility, a number of trays at the facility and needed to be sent through the sterilizer, and a number of trays at the facility and sent through the sterilizer. These numbers are updated as trays are at the facility and sent through the sterilizer, as described herein.

The interfaces of FIGS. 29-31 may be accessed using the dashboard user interface 2800 shown in FIG. 28.

FIG. 32 illustrates an exemplary mobile application user interface 3200 displaying the invocation of a mobile phone scanning software invoked by the camera icon as shown in FIG. 28 for scanning visual identifiers (e.g., a barcode) to check out trays or check in trays, according to an exemplary embodiment.

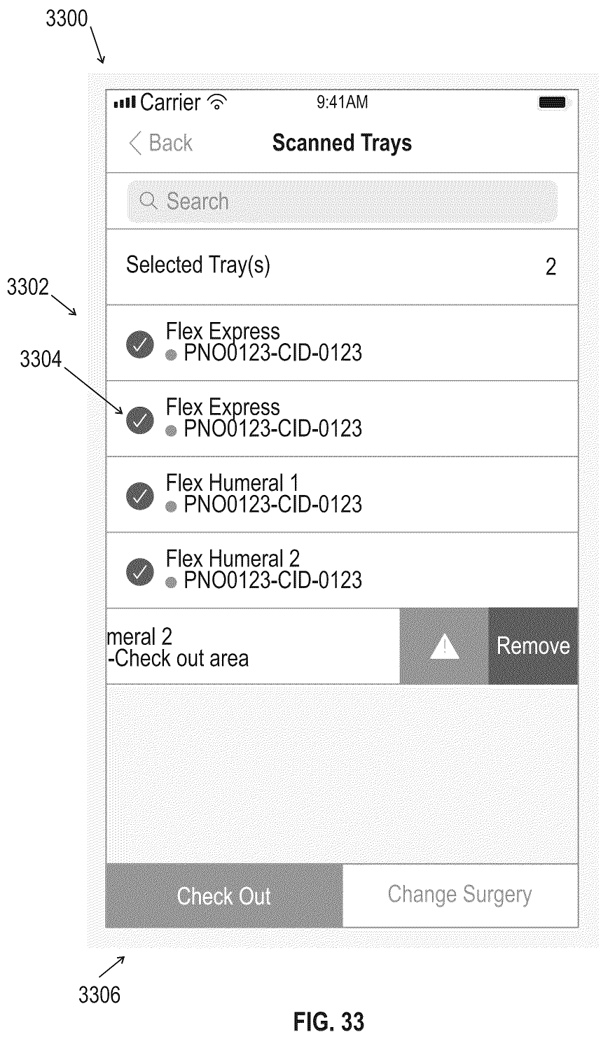

In some embodiments, one or more trays include barcodes (such as a hardened sticker) attached to an inside or an outside of the tray. The mobile application user interface 3200 uses a camera on the mobile device to scan the visual identifier. The view of the camera is shown in box 3202 for scanning the visual identifier. In some embodiments, once the visual identifier is scanned, the tray is checked in at the facility and is ready for processing. In another embodiment, once one or more visual identifiers are scanned, the user is shown mobile application user interface 3300 of FIG. 33 displaying a list of all of the trays scans which were just scanned.

In some embodiments, when checking in a tray, a label is generated. That label will have a barcode on it. Interface 3200 enables a user to scan the barcode on these labels to quickly identify the tray for check out. The number 3204 will increase with every scan. The user then needs to click the number 3204 to be brought to interface 3300 in FIG. 33.

The user may use mobile application user interface 3200 to successively scan visual identifiers on one or more trays, thereby quickly checking in or checking out, the one or more trays. In the illustrated embodiment, the user scanned 5 trays indicated by icon 3204, and as shown in FIG. 33.

In some embodiments, the user may use mobile application user interface 3200 to successively scan visual identifiers on one or more trays and assign the one or more trays to another surgery.

In some embodiments, interface 3200 can be used to scan a barcode on a tag. This scan, will quickly identify the tag associated with each tray and therefore make the check in process faster.

FIG. 33 illustrates an exemplary mobile application user interface 3300 displaying a list of scanned trays 3302, according to an exemplary embodiment. In an exemplary embodiment, the trays were scanned using the mobile application user interface 3200 shown in FIG. 32. A representative can further use mobile application user interface 3300 to check out trays. The representative may select one or more trays via selectable circles 3304 associated with the trays, and “checkout” the selected trays using the checkout button 3306. The representative checks out the tray to break the link of the tag from tray.

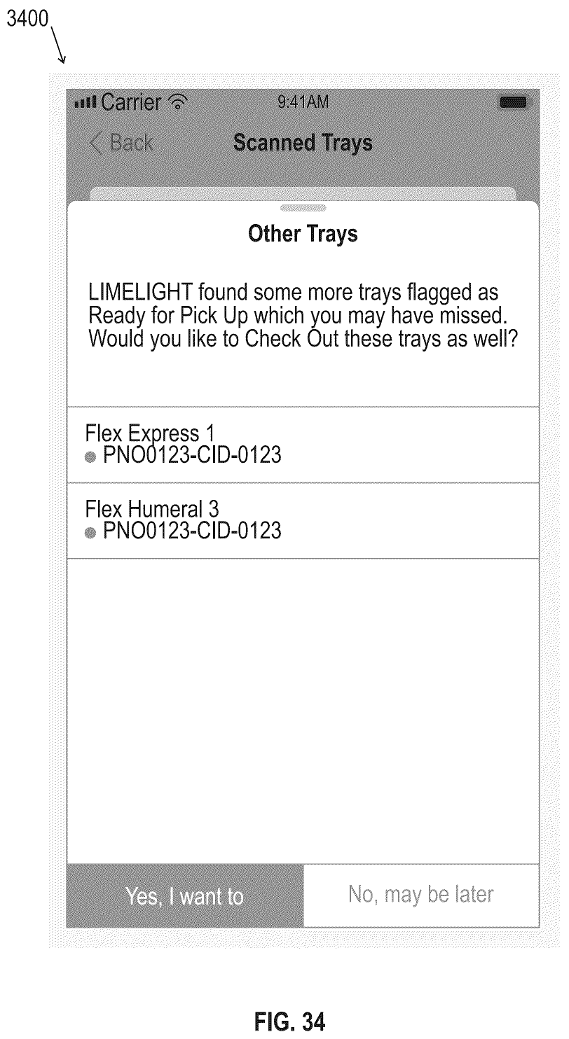

FIG. 34 illustrates an exemplary mobile application user interface 3400 displaying a notification or reminder that there are additional trays ready for pick up from the facility and/or to be checked out, according to an exemplary embodiment.

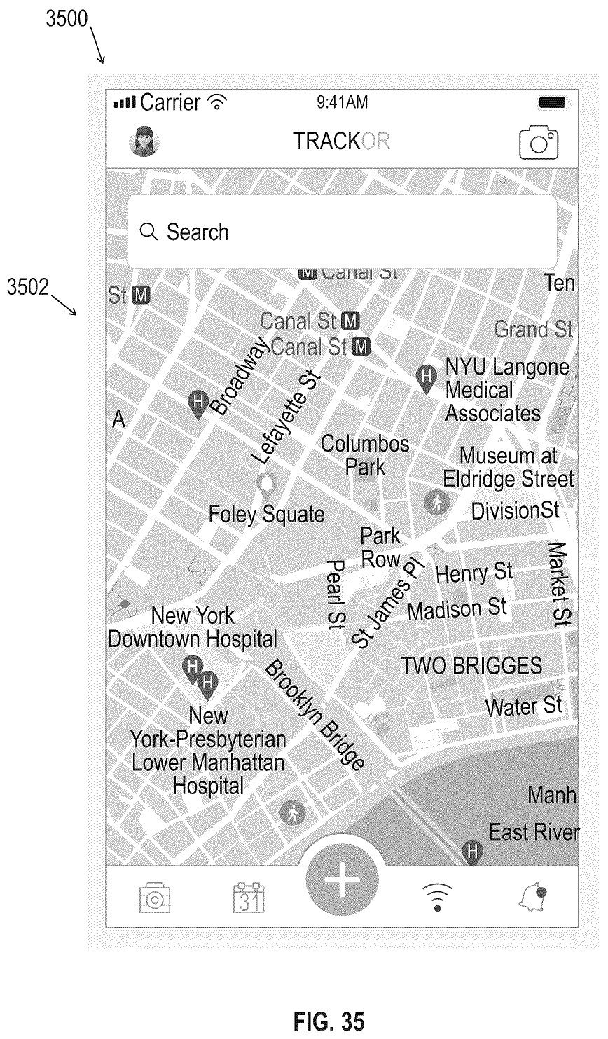

FIG. 35 illustrates an exemplary mobile application user interface 3500 displaying a map 3502 of locations of facilities associated with trays, according to an exemplary embodiment. In one embodiment, the map 3502 displays one or more locations of facilities that require deliveries of trays for upcoming surgeries. In another embodiment, the map 3502 displays one or more locations of facilities that require a retrievals of trays post-surgeries.

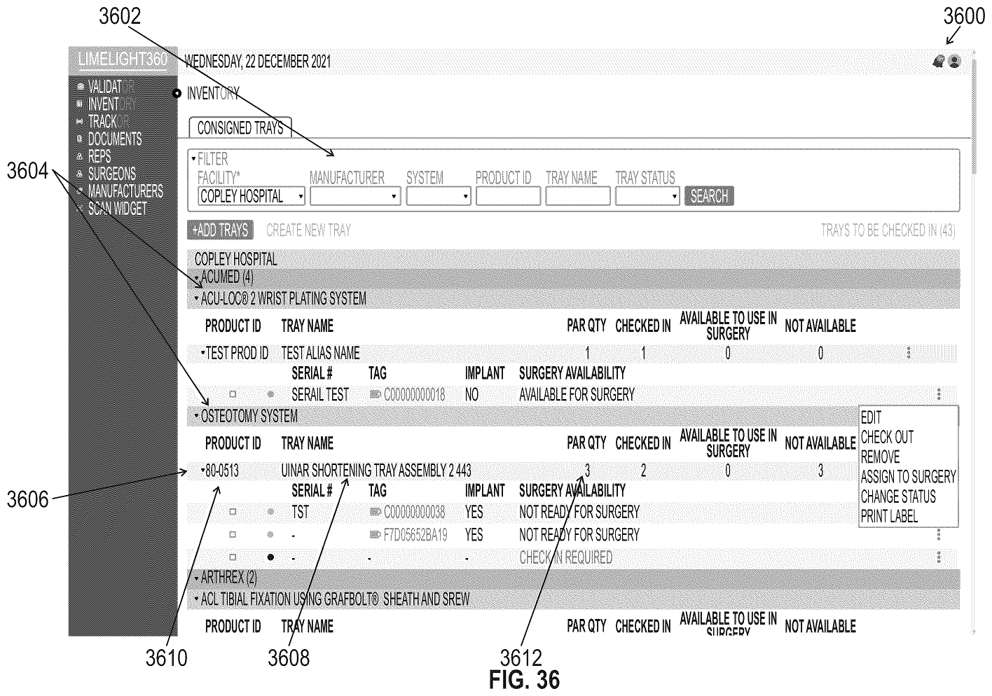

FIGS. 36-49 illustrate embodiments in which one or more trays are on consignment within a facility and/or are available for turnover. Trays on consignment are located within the facility for a period of time. In consignment, a vendor leaves trays with the facility without transferring ownership to the facility. These trays are constantly reprocessed and used for surgeries within the facility. Trays available for turnover can be reprocessed and reused for upcoming surgeries.

FIG. 36 illustrates an exemplary dashboard user interface 3600 for viewing consigned trays, according to an exemplary embodiment. A user can use panel 3602 to filter the consigned trays based on particular variables. The results can be filtered based on facility, manufacturer, system, product identifier, tray name, and/or tray status. In exemplary dashboard user interface 3600, the results are filtered by facility “Copley Hospital.”

In the illustrated embodiment, dashboard user interface 3600 displays consigned trays within the facility based on categories 3604. In the illustrated embodiment, the categories 3604 include Acu-Loc® Wrist Plating System, Osteotomy System, Arthrex, and ACL Tibial Fixation using GraftBolt® Sheath and Screw. However, other embodiments can include different categories.

Each category 3604 may include information for one or more consigned trays 3606. Each consigned tray 3606 includes a tray name 3608 identifying particular tray component within the tray. For example, a tray name may be “ulnar shortening tray assembly,” which identifies that the tray includes components for an ulnar shortening osteotomy. Each consigned tray 3606 further includes may include a product identifier 3610, and/or a quantity of trays on consignment 3612. The quantity of trays on consignment 3612 includes a number of trays checked in, a number of trays available to use in surgery, and a number of trays not available. Each tray of the quantity of trays on consignment 3612 includes one or more of a color coded indicator indicating whether the tray is ready for surgery, a serial number, a tag number, whether the tray includes an implant, and whether the tray is ready for surgery.

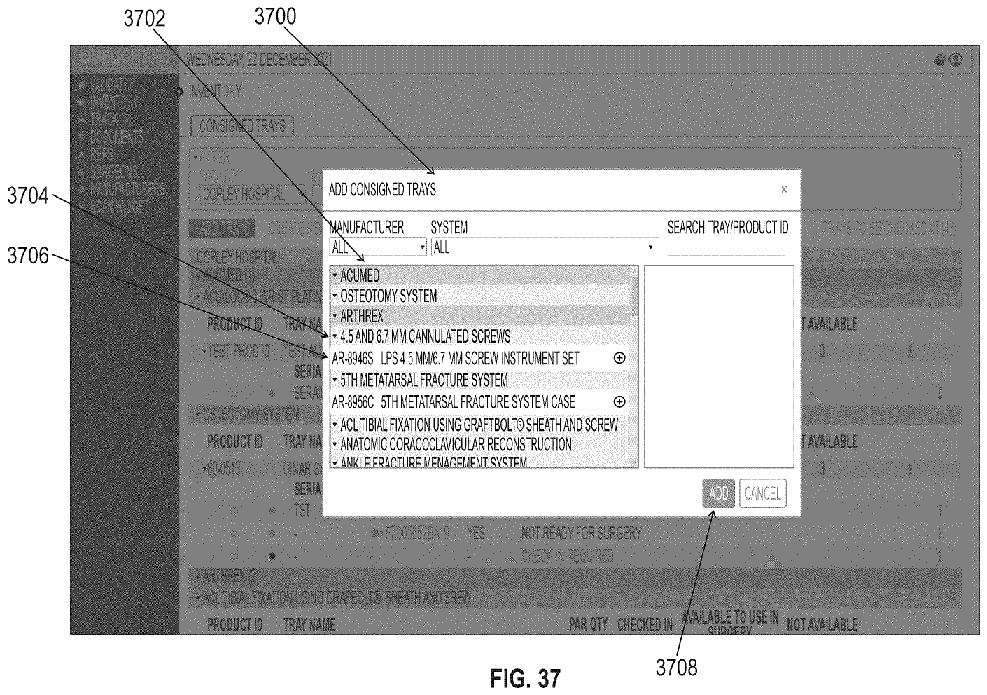

FIG. 37 illustrates an exemplary user interface 3700 for adding a consigned tray, according to an exemplary embodiment. A user can make a tray selection based on categories. The user can further filter trays based on manufacturers, systems, and/or a product identifier. A user may select a category 3704, then a tray name and/or product identifier 3706, to select a tray to add as a consignment. The user selects an “Add” button 3708 to add the tray as a consignment. The added consigned tray will appear in the dashboard user interface 3600 as shown in FIG. 36.

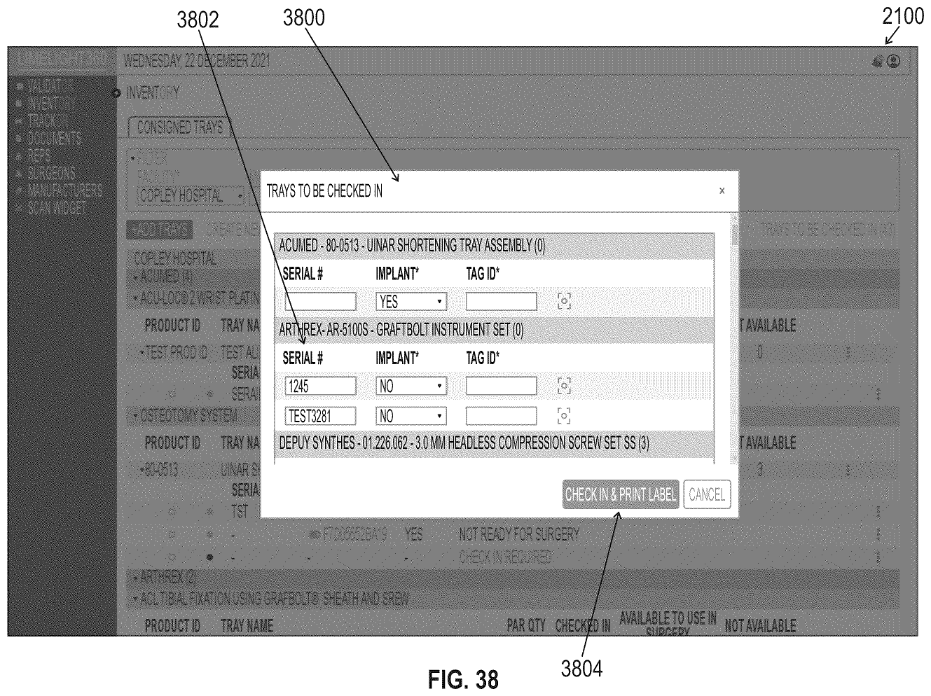

FIG. 38 illustrates an exemplary user interface 3800 for checking in a consigned tray, according to an exemplary embodiment. A user enters information regarding the consigned tray 3802, such as a serial identifier, whether the tray includes an implant, and a tag identifier. The user selects a “Check In & Print Label” button 3804, whereupon the consigned tray is checked in and a label is printed for the consigned tray.

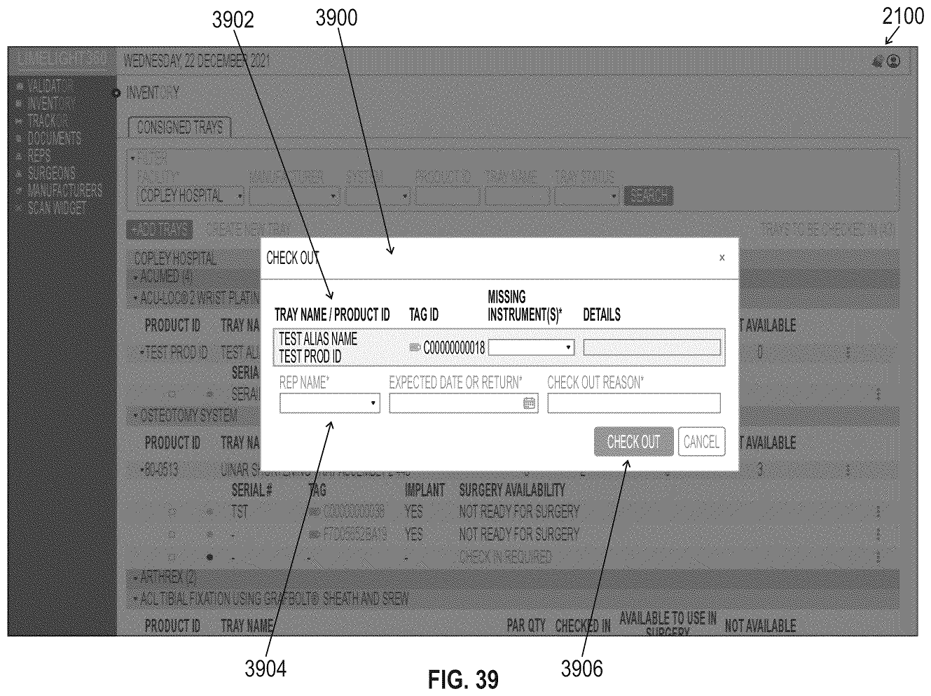

FIG. 39 illustrates an exemplary user interface 3900 for checking out a consigned tray, according to an exemplary embodiment. Information regarding the consigned tray 3902 being check out is displayed in interface 3900, such as a tray name, a product identifier, and/or a tag identifier for the consigned tray being checked out. A user enters information regarding the checkout 3904, such as a representative name, expected date of return of the consigned tray, missing instruments from the consigned tray, and a reason for checking out the consigned tray. The user selects a “Check Out” button 3906, whereupon the consigned tray is checked in and a label is printed for the consigned tray.

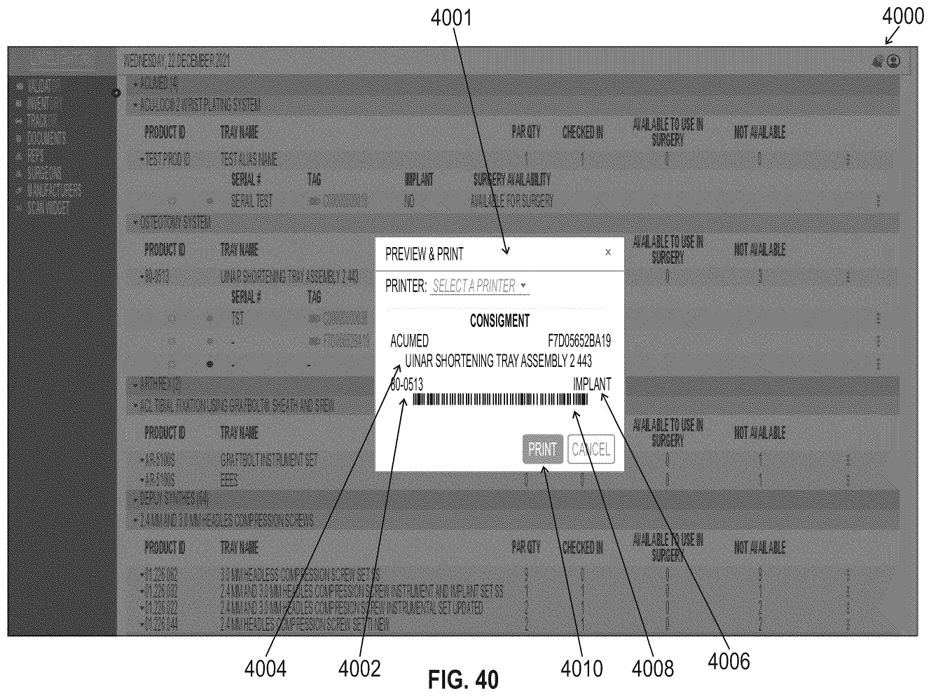

FIG. 40 illustrates an exemplary user interface 4000 for reviewing and printing a label 4001 after checking in a consigned tray, according to an exemplary embodiment. In the exemplary embodiment, the label 4001 includes a product identifier 4002, a tray name 4004, whether the tray includes an implant 4006, and a barcode 4008 for scanning the tray to check in, check out, and/or reassign the tray. A user can select a “Print” button to send the label to a printer. The physical label can then be attached to the tray.

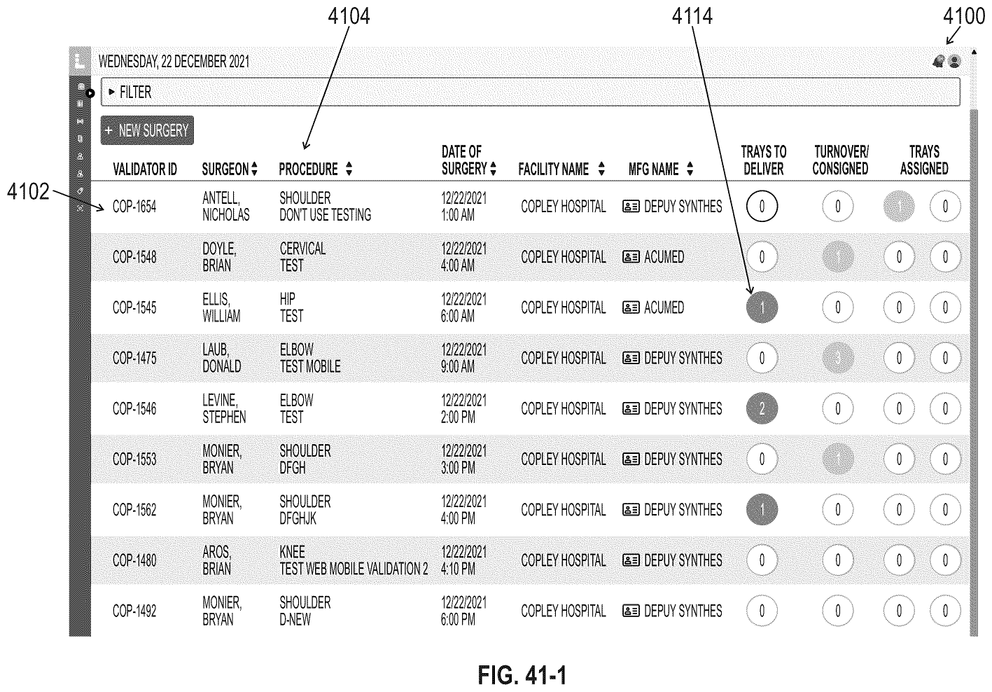

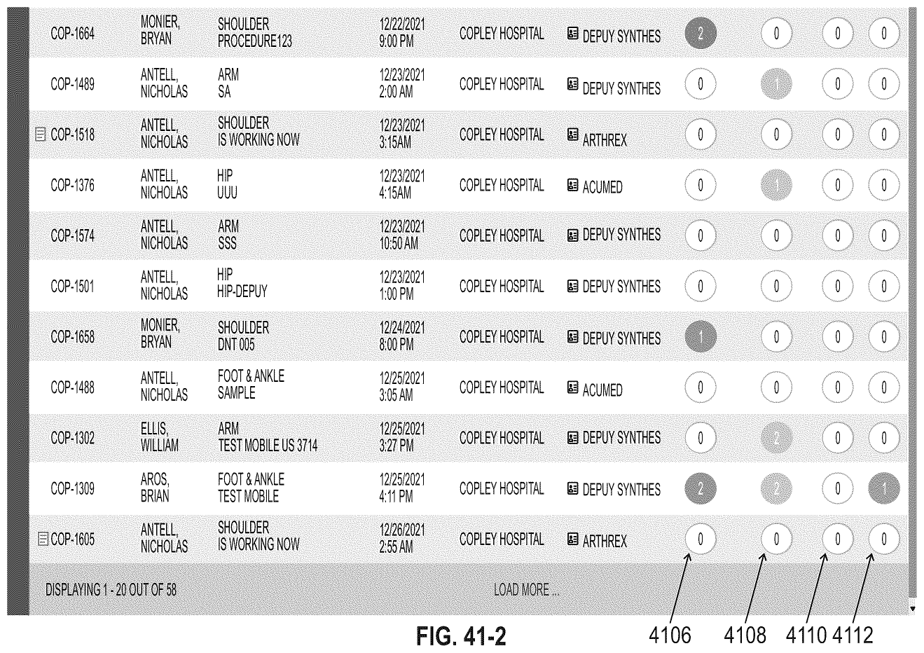

FIG. 41 illustrates an exemplary pre-operative dashboard user interface 4100 displaying a list of surgeries that require one or more surgical trays, according to an exemplary embodiment. In some embodiments, the interface 4100 enables medical/surgical representatives as well as facility employees to manage medical device trays.

The interface 4100 includes a list of entries 4102. Each entry 4102 is associated with a surgery 4104 and a number 4106 of trays required for the surgery but have not been received by the facility. Each entry 4102 further includes a number 4108 of consigned trays and turn over trays that are available for the procedure. Each entry 4102 further includes a number 4110 of trays assigned to a surgery but not yet sent through the sterilizer and ready for surgery. Each entry 4102 further includes a number of 4112 of trays required for a surgery and that have been through the sterilizer and are ready for surgery. The numbers 4106, 4108, 4110, and 4112 are updated as trays are received by the facility, added as consigned or on turn over, assigned, and/or sent through the sterilizer for surgery.

In some embodiments, each number 4106, 4108, 4110, and 4112 is associated with a color (e.g., green, yellow, red, gray, and blue). For example, the number 304 of trays required for a surgery but not been received by the facility are associated with the color blue or red, depending on the time of delivery in comparison to the delivery policies set forth by the facility. In the exemplary embodiment, this number 4114 appears in the first number column and is appropriately color-coded, such as in a red circle, since the delivery is in violation of the facility's policy.

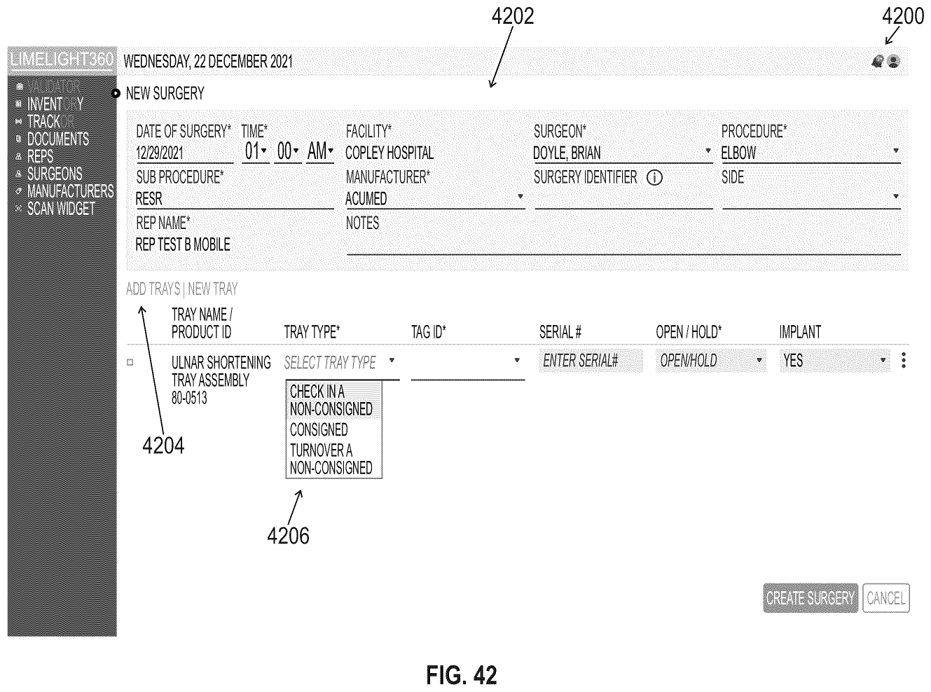

FIG. 42 illustrates an exemplary pre-operative dashboard user interface 4200 to add a new surgery, according to an exemplary embodiment. In dashboard user interface 4200, a user, such as a representative, may enter information about a new surgery into panel 4202, such as, but not limited to, a name of a facility where the surgery is being performed, a name of a surgeon performing the surgery, a name of a surgery/procedure and sub-surgery/procedure, a surgery date and time, a name of a representative, a name of a manufacturer associated with the tray(s), and a side of the surgery (e.g., left side or right side). In some embodiments, this information may be selectable via drop down menus. For example, in some embodiments, each drop down menu may display particular options, for example, options associated with the representative, the surgery, or the surgeon. For example, a representative may only be associated with one or more particular manufacturers or particular facilities.

In some embodiments, the user may add trays to the surgery using link 4204. For example, when a surgery is booked, the representative identifies the trays needed to support the surgery and uses interface 4200 to enter the trays. For each tray, the user includes a tray type 4206. The tray type 4206 describes whether the added tray is a non-consigned tray, a consigned tray, or is a turnover of a non-consigned tray. The tray type 4206 options includes to “check in in a non-consigned tray,” “a consigned tray,” or “turnover a non-consigned tray.” Of note, the selected designation for each tray drives the representation in FIG. 41, columns 4106 and 4108.

Once the new surgery is created, and optionally the trays are added to the new surgery, a new entry is created in the pre-operative dashboard user interface 4100.

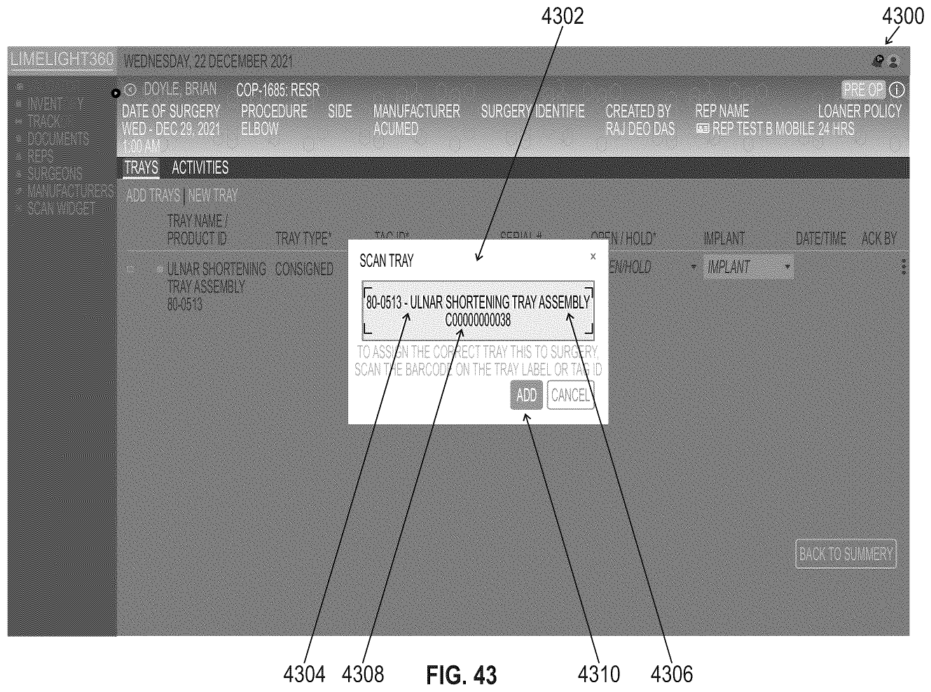

FIG. 43 illustrates an exemplary user interface 4300 to add a tray to a surgery by scanning a visual identifier (e.g., a barcode) associated with the tray, according to an exemplary embodiment. Panel 4302 illustrates an exemplary user interface displaying scanning software for scanning a barcode on a tray label associated with the tray to obtain a product identifier and/or a product name, or for scanning a bar code on a tag to obtain a tag identifier, to add the tray to the surgery. Panel 4302 depicts a product identifier 4304, a product name 4306, and a tag identifier 4308 obtained from scanning the barcode. The user selects an “Add” button 4310 to add the tray to the surgery.

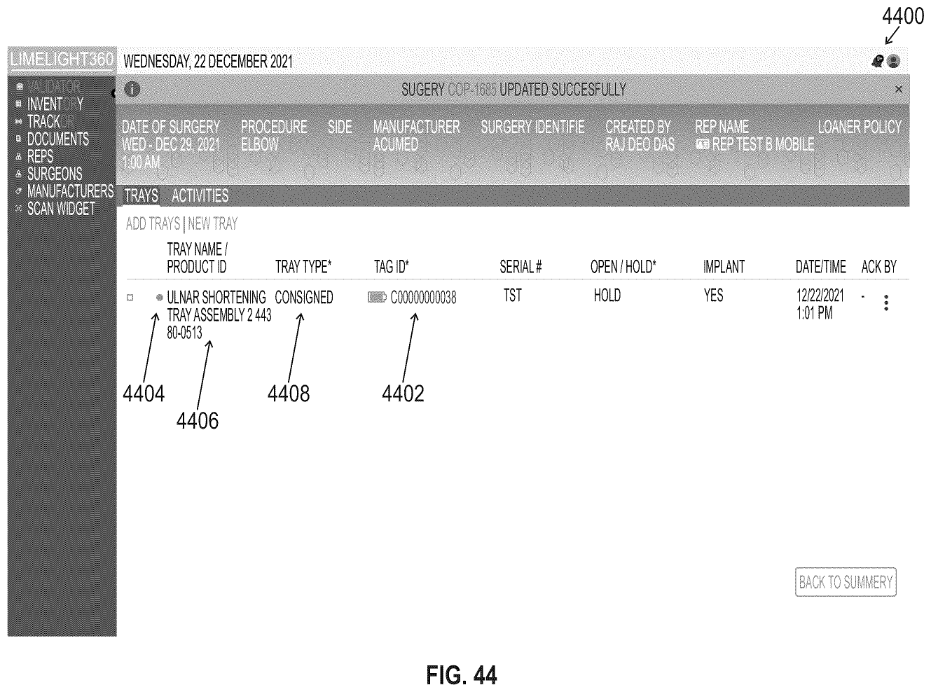

FIG. 44 illustrates an exemplary dashboard user interface 4400 after a tray is added to a surgery, according to an exemplary embodiment. In the illustrated embodiment, the tray is associated with a tag, as shown at 4402. As illustrated, the tray information includes, but is not limited to, a color coded identifier 4404 identifying whether the tray is ready for surgery, a tray name and product identifier 4406, a tray type 4408 (e.g., “consigned”), and the tag identifier 4402.

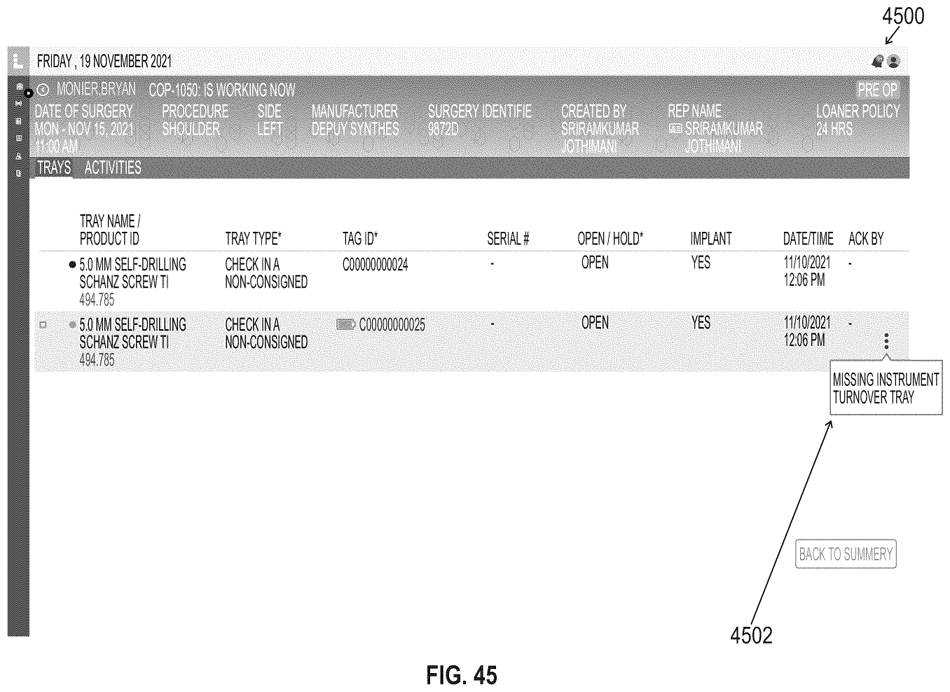

FIG. 45 illustrates an exemplary dashboard user interface 4500 illustrating an option 4502 to turn over a tray, according to an exemplary embodiment. Turning over a tray enables a non-consigned tray to be used in another surgery. The user selects a “Turnover Tray” option 4502 to turn over the tray for another surgery.

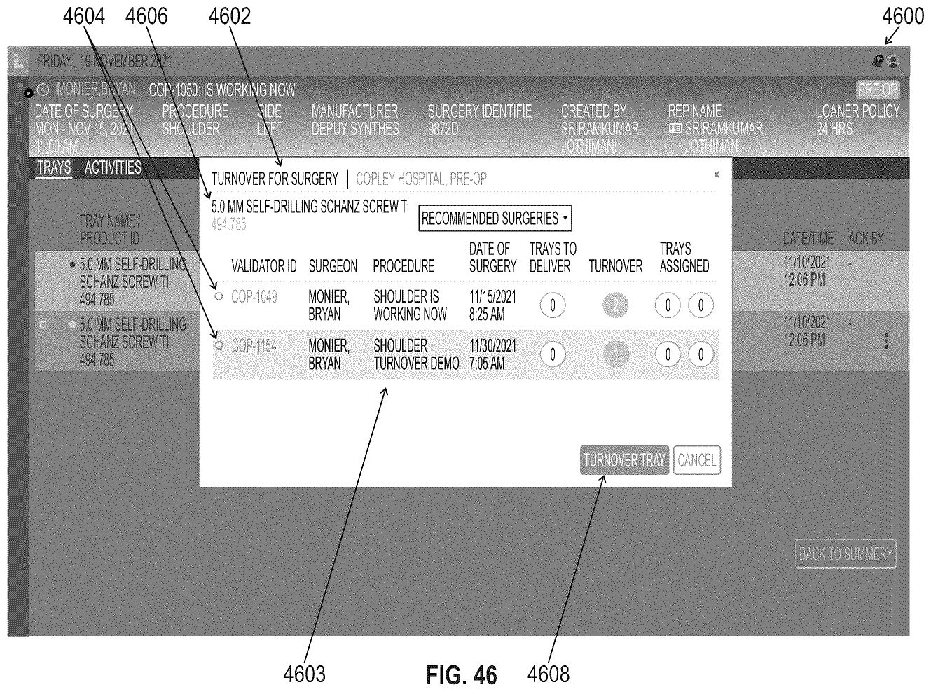

FIG. 46 illustrates an exemplary user interface 4600 for turning over a tray, according to an exemplary embodiment. For example, after selecting the “Turnover Tray” option 4502 in FIG. 45, window 4602 is displayed to the user for selecting an upcoming surgery to receive the turn over tray. Window 4603 displays a list of upcoming surgeries that may be appropriate for the turn over tray. Window 4602 displays information 4606 on the turn over tray, such as a tray name. The user can filter and/or arrange the list of upcoming surgeries by, for example, recommended surgeries or surgery dates. Each upcoming surgery include information on the upcoming surgery, such as a surgeon name, a surgery name, and a date of surgery, The user can select 4604 an upcoming surgery to receive the turn over tray, and select a “Turnover Tray” button 4608 to turn over the tray for the selected surgery.

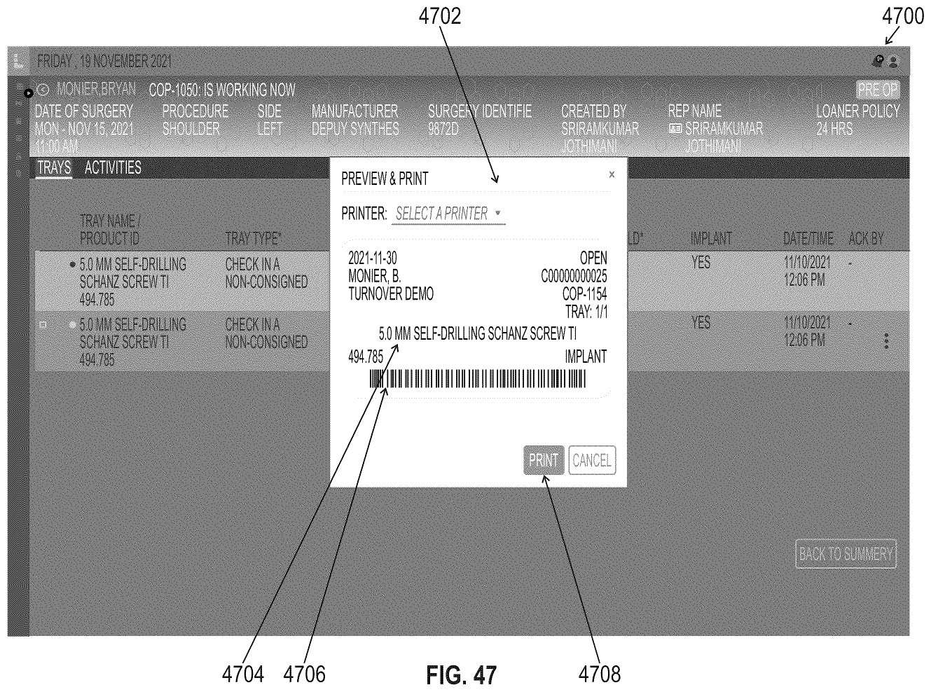

FIG. 47 illustrates an exemplary user interface 4700 for reviewing and printing a label after turning over a tray, according to an exemplary embodiment. In the exemplary embodiment, the label 4702 includes information on the turn over tray, such as, but not limited to, a tray name 4704 and a barcode 4706 for scanning the turn over tray to check in, check out, and/or reassign the turn over tray. A user can select a “Print” button 4708 to send the label to a printer. The physical label can then be attached to the turn over tray.

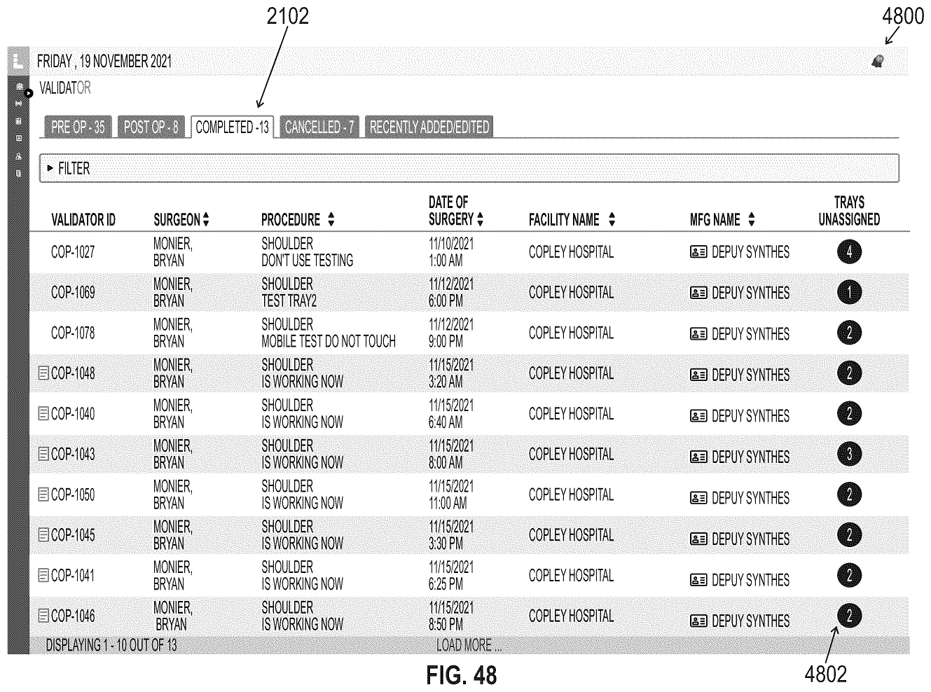

FIG. 48 illustrates an exemplary user interface 4800 displaying a list 4802 of unassigned trays for completed surgeries, according to an exemplary embodiment.

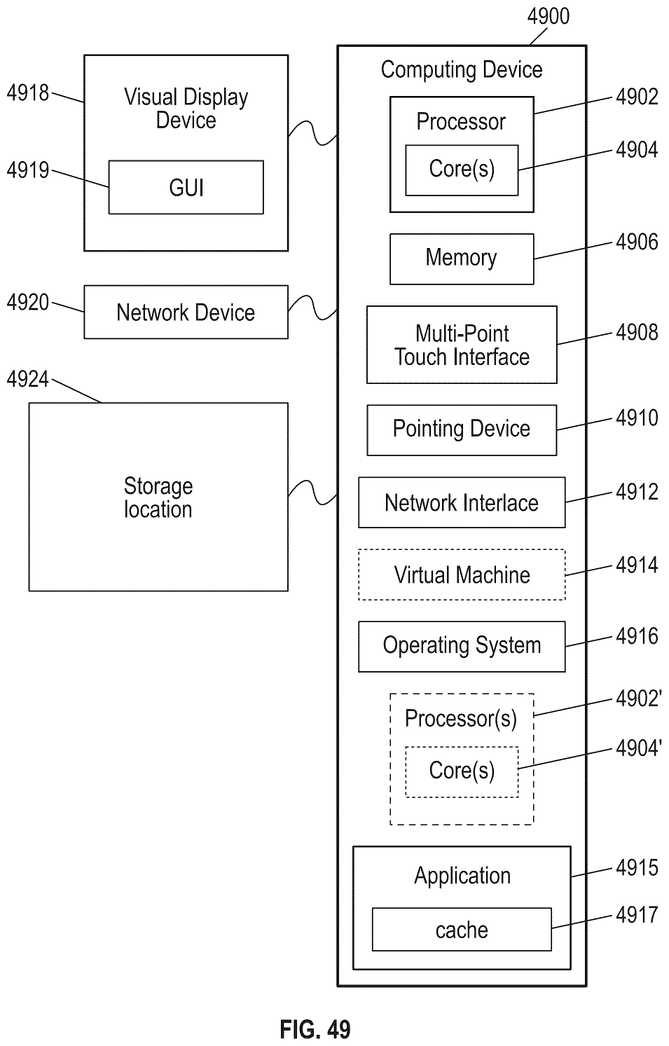

FIG. 49 is a block diagram of an example computing device 4900 that can be used to perform one or more steps of the systems and methods provided by exemplary embodiments. In an exemplary embodiment, computing device 4900 is IoT computing device 112 and/or client device 113 illustrated in FIG. 1. Computing device 4900 includes one or more processors 4902. Computing device 4900 includes one or more non-transitory computer-readable media for storing one or more computer-executable instructions or software for implementing exemplary embodiments such as the prioritization module described herein. The non-transitory computer-readable media can include, but are not limited to, one or more types of hardware memory, non-transitory tangible media (for example, one or more magnetic storage disks, one or more optical disks, one or more USB flash drives), and the like. For example, a memory 4906 included in computing device 4900 can store computer-readable and computer-executable instructions or software for implementing exemplary embodiments. Computing device 4900 also includes a core 4904 associated with processor 4902, and optionally, one or more additional processor(s) 4902′ and associated core(s) 4904′ (for example, in the case of computer systems having multiple processors/cores), for executing computer-readable and computer-executable instructions or software stored in memory 4906 and other programs for controlling system hardware. Processor 4902 and processor(s) 4902′ can each be a single core processor or multiple core (4904 and 4904′) processor. Computing device 4900 may include an browser application 4915 and a browser cache 4917. As described above, the browser application 4915 can enable a user to view user interfaces.

Virtualization can be employed in computing device 4900 so that infrastructure and resources in the computing device can be shared dynamically. A virtual machine 4914 can be provided to handle a process running on multiple processors so that the process appears to be using only one computing resource rather than multiple computing resources. Multiple virtual machines can also be used with one processor.

Memory 4906 can include a computer system memory or random access memory, such as DRAM, SRAM, EDO RAM, and the like. Memory 4906 can include other types of memory as well, or combinations thereof. In some embodiments, a user can interact with computing device 4900 through a visual display device 4918, such as a touch screen display or computer monitor, which can display one or more user interfaces. Visual display device 4918 may also display other aspects, elements and/or information or data associated with exemplary embodiments. Computing device 4900 may include other I/O devices for receiving input from a user, for example, a keyboard or any suitable multi-point touch interface 4908, a pointing device 4910 (e.g., a pen, stylus, mouse, or trackpad). The keyboard 4908 and pointing device 4910 may be coupled to visual display device 4918. Computing device 4900 may include other suitable conventional I/O peripherals.

Computing device 4900 can also include one or more storage devices 4924, such as a hard-drive, CD-ROM, or other computer readable media, for storing data and computer-readable instructions and/or software that implements embodiments of the system, as described herein, or portions thereof. Exemplary storage device 4924 can also store one or more storage devices for storing any suitable information required to implement exemplary embodiments. For example, storage device 4924 and/or memory 4906 store portions of data and/or files.

Computing device 4900 can include a network interface 4912 configured to interface via one or more network devices 4922 with one or more networks, for example, Local Area Network (LAN), Wide Area Network (WAN) or the Internet through a variety of connections including, but not limited to, standard telephone lines, LAN or WAN links (for example, 802.11, T1, T3, 56kb, X.25), broadband connections (for example, ISDN, Frame Relay, ATM), wireless connections, controller area network (CAN), or some combination of any or all of the above. The network interface 4912 can include a built-in network adapter, network interface card, PCMCIA network card, card bus network adapter, wireless network adapter, USB network adapter, modem or any other device suitable for interfacing computing device 4900 to any type of network capable of communication and performing the operations described herein. Moreover, computing device 4900 can be any computer system, such as a workstation, desktop computer, storage server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

Computing device 4900 can run any operating system 4916, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In exemplary embodiments, the operating system 4916 can be run in native mode or emulated mode. In an exemplary embodiment, the operating system 4916 can be run on one or more cloud machine instances.

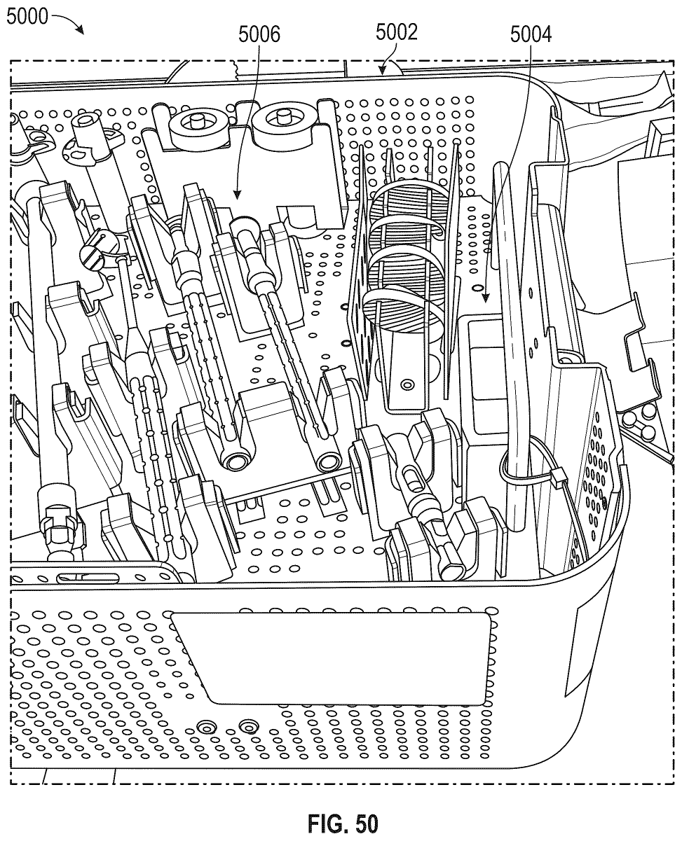

FIG. 50 is a photograph 5000 of a tray 5002 containing a tag 5004 located within the tray 5002, in accordance with an exemplary embodiment. The tray 5002 further includes one or more medical instruments 5006 for a surgery. The tray 5002 is designed to undergo sterilization within an autoclave. The tag 5004 includes a hardened shell to survive repeated autoclave cycles while not deteriorating in longevity or performance as a result of the rigors of the autoclave cycles.

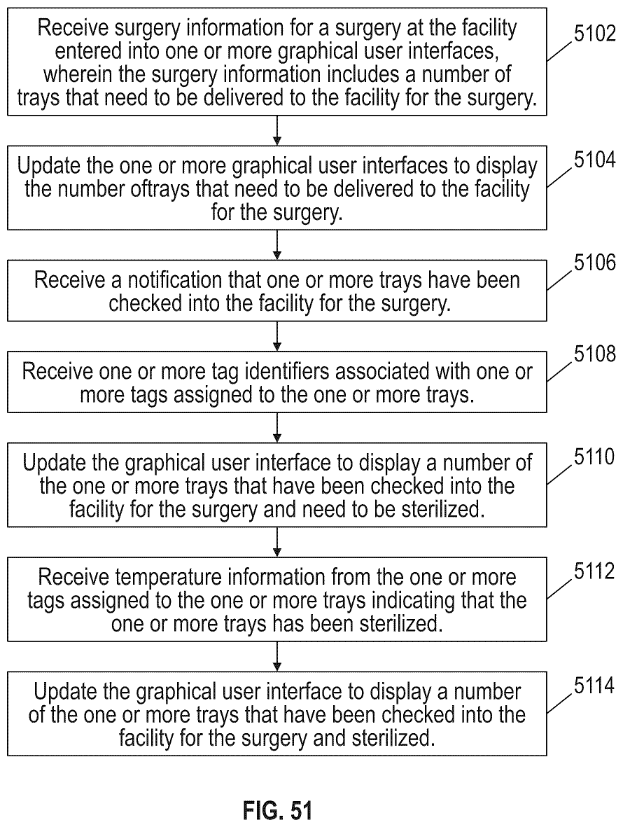

FIG. 51 is a flow chart of an example process that can be executed in accordance with some embodiments of the present disclosure. Step 5102 includes receiving surgery information for a surgery at the facility entered into one or more graphical user interfaces, wherein the surgery information includes a number of trays that need to be sourced to the facility for the surgery. Step 5104 includes updating the one or more graphical user interfaces to display the number of trays that need to be sourced to the facility for the surgery. Step 5106 includes receiving a notification that one or more trays have been checked in at the facility for the surgery. Step 5108 includes receiving one or more tag identifiers associated with one or more tags assigned to the one or more trays. Step 5110 includes updating the graphical user interface to display a number of the one or more trays that have been checked in at the facility for the surgery and need to be sent through the sterilizer. Step 5112 includes receiving temperature information from the one or more tags assigned to the one or more trays indicating that the one or more trays have been sent through the sterilizer. Step 5114 includes updating the graphical user interface to display a number of the one or more trays that have been checked in at the facility for the surgery and sent through the sterilizer.

The description herein is presented to enable any person skilled in the art to create and use a computer system configuration and related method and systems for generating an improved unique device identifier (UDI) for medical devices and tracking of medical instruments within a facility and location-based validation of autoclave parametric sterilization release. Various modifications to the example embodiments will be readily apparent to those skilled in the art, and the generic principles defined herein may be applied to other embodiments and applications without departing from the spirit and scope of an exemplary embodiment. Moreover, in the following description, numerous details are set forth for the purpose of explanation. However, one of ordinary skill in the art will realize that embodiments of the present disclosure may be practiced without the use of these specific details. In other instances, well-known structures and processes are shown in block diagram form in order not to obscure the description of embodiments of the present disclosure with unnecessary detail. Thus, an exemplary embodiment is not intended to be limited to the embodiments shown, but is to be accorded the widest scope consistent with the principles and features disclosed herein.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements, device components or method steps, those elements, components or steps can be replaced with a single element, component, or step. Likewise, a single element, component, or step can be replaced with a plurality of elements, components, or steps that serve the same purpose. Moreover, while exemplary embodiments have been shown and described with references to particular embodiments thereof, those of ordinary skill in the art will understand that various substitutions and alterations in form and detail can be made therein without departing from the scope of an exemplary embodiment. Further still, other aspects, functions, and advantages are also within the scope of an exemplary embodiment.

Exemplary flowcharts are provided herein for illustrative purposes and are non-limiting examples of methods. One of ordinary skill in the art will recognize that exemplary methods can include more or fewer steps than those illustrated in the exemplary flowcharts, and that the steps in the exemplary flowcharts can be performed in a different order than the order shown in the illustrative flowcharts.

We claim:

1. A system for medical instrument management comprising:
one or more graphical user interfaces displaying one or more upcoming surgeries at a facility, wherein for each surgery of the one or more upcoming surgeries, the one or more graphical user interfaces display:
a number of trays needed to be sourced for a surgery,
a number of trays sourced for the surgery, located at the facility, and needed to be sent through a sterilizer, and
a number of trays sourced for the surgery, located at the facility, and sent through the sterilizer; and
one or more tags configured to transmit location information; and
one or more processors configured to:
receive surgery information for an upcoming surgery at the facility entered into the one or more graphical user interfaces, wherein the surgery information includes a number of trays that need to be sourced for the upcoming surgery;
update the one or more graphical user interfaces to display the number of trays that need to be sourced for the upcoming surgery;
receive a notification that one or more trays have been sourced for the upcoming surgery;
receive one or more tag identifiers associated with the one or more tags assigned to the one or more trays;
update the graphical user interface to display a number of the one or more trays that have been sourced for the upcoming surgery and need to be sent through the sterilizer;
receive temperature information from the one or more tags assigned to the one or more trays indicating that the one or more trays have been sent through the sterilizer; and
update the graphical user interface to display a number of the one or more trays that have been sourced for the upcoming surgery and sent through the sterilizer.

2. The system of claim 1, further comprising:
a second graphical user interface displaying a number of trays ready to be retrieved from the facility,
wherein the one or more processors are further configured to:
receive a notification that a link between the tag identifier associated with the tag and a tray of the one or more trays have been disassociated, wherein the notification indicates that the tray is ready to be retrieved from the facility; and
update the second graphical user interface to display an updated number of trays ready to be retrieved from the facility.

3. The system of claim 2, further comprising:
the second graphical user interface displaying a number of trays that have been retrieved from the facility,
wherein the one or more processors are further configured to:
receive a notification that a tray has been retrieved from the facility; and
update the second graphical user interface to display an updated number of trays retrieved from the facility.

4. The system of claim 1, wherein the surgery information further comprises one or more of a surgery identifier, a name of a surgeon performing the upcoming surgery, a name of a procedure, a date of the upcoming surgery, and a name of a manufacturer associated with the one or more trays.

5. The system of claim 1, wherein the number of trays needed to be sourced to the facility is associated with a first color, the number of trays at the facility and needed to be sent through the sterilizer is associated with a second color, and the number of trays at the facility and sent through the sterilizer is associated with a third color.

6. The system of claim 1, wherein the one or more processors are further configured to receive location information from the tag assigned to the tray indicating the location of the tray.

7. The system of claim 1, further comprising:
a second graphical user interface displaying a progress bar configured to display locations associated with a tray of the one or more trays,
wherein the one or more processors are further configured to update the progress bar based on a location of a tag of the one or more tags communicating with beacons located in different rooms.

8. The system of claim 1, wherein trays are sourced from medical tray suppliers delivering the trays to the facility, from re-using consigned trays, or from turning over trays.

9. The system of claim 1, wherein the one or more tags are near field communication devices and configured to transmit location information by communicating with two or more beacons, each beacon located in a different area.

10. The system of claim 1, further comprising:
a second graphical user interface displaying a map of the facility displaying a location of the tray on the map based on a location of a tag of the one or more tags.

11. A method for medical instrument management comprising:
receiving surgery information for a surgery at the facility entered into one or more graphical user interfaces, wherein the surgery information includes a number of trays needed to be sourced to the facility for the surgery;
updating the one or more graphical user interfaces to display the number of trays that need to be sourced for the surgery;
receiving a notification that one or more trays have been sourced for the surgery;
receiving one or more tag identifiers associated with one or more tags assigned to the one or more trays;
updating the graphical user interface to display a number of the one or more trays that have been sourced for the surgery and need to be sent through a sterilizer;
receiving temperature information from the one or more tags assigned to the one or more trays indicating that the one or more trays have been sent through the sterilizer; and
updating the graphical user interface to display a number of the one or more trays that have been sourced for the surgery and sent through the sterilizer.

12. The method of claim 11, further comprising:
receiving a notification that a link between the tag identifier associated with the tag and a tray of the one or more trays have been disassociated, wherein the notification indicates that the tray is ready to be retrieved from the facility; and
updating a second graphical user interface displaying a number of trays ready to be retrieved from the facility to display an updated number of trays ready to be retrieved from the facility.

13. The method of claim 12, further comprising:
receiving a notification that a tray of the one or more trays have been retrieved from the facility; and
updating the second graphical user interface displaying a number of trays that have been retrieved from the facility to display an updated number of trays retrieved from the facility.

14. The method of claim 11, wherein the surgery information further comprises wherein the surgery information further comprises one or more of a surgery identifier, a name of a surgeon performing the surgery, a name of a procedure, a date of the surgery, and a name of a manufacturer associated with the one or more trays.

15. The method of claim 11, further comprising associating the number of trays needed to be sourced to the facility with a first color, associating the number of trays at the facility and needed to be sent through the sterilizer with a second color, and associating the number of trays at the facility and sent through the sterilizer with a third color.

16. The method of claim 11, further comprising:
receiving location information from the one or more tags assigned to the one or more trays indicating locations of the one or more trays.

17. The method of claim 11, further comprising:
displaying a progress bar via a second graphical user interface configured to display locations associated with a tray of the one or more trays; and
updating the progress bar based on a location of a tag of the one or more tags communicating with one or more beacons.

18. The method of claim 11, wherein trays are sourced from medical tray suppliers delivering the trays to the facility, from re-using consigned trays, or from turning over trays.

19. The method of claim 11, transmitting, via the one or more tags, location information to a beacon of two or more beacons, each beacon located in a different area.

20. The method of claim 11, further comprising:
displaying a map of the facility on a second graphical user interface, the map displaying a location of the tray on the map based on a location of a tag of the one or more tags.
